(19) United States
(12) Patent Application Publication
(10) Pub. No.: US 2026/0250308 A1
(43) Pub. Date: Aug. 27, 2026

(54) ZWITTERIONIC METAL CHELATORS

(71) Applicant: Curadel Surgical Innovations, Inc., Natick, MA (US)

(72) Inventors: John V. Frangioni, Weston, MA (US); Wolfgang Maison, Winsen (DE); Deepak Suresh Sail, Bonita Springs, FL (US)

(73) Assignee: Curadel Surgical Innovations, Inc., Natick, MA (US)

(21) Appl. No.: 19/394,299

(22) Filed: Nov. 19, 2025

Related U.S. Application Data
(60) Provisional application No. 63/724,048, filed on Nov. 22, 2024.
(60) Provisional application No. 63/724,707, filed on Nov. 25, 2024.
(60) Provisional application No. 63/767,817, filed on Mar. 6, 2025.

(51) Int. Cl. C07F 15/00; A61K 51/04

(52) U.S. Cl. CPC C07F 15/00; A61K 51/0482

(57) ABSTRACT

The present invention relates to zwitterionic metal chelators and their use as imaging, diagnostic, chemical processing, and treatment agents. These zwitterionic metal chelators have desirable properties that maximize solubility in aqueous environments, minimize non-specific interactions, and retain the ability to target thus resulting in an improved performance in a variety of medical, agricultural, and chemical processes. In in vivo and medical applications, zwitterionic metal chelators improve the signal-to-background ratio and therapeutic window as compared to other metal chelators while retaining high stability.

### CROSS-REFERENCE TO RELATED APPLICATIONS

This application claims priority to U.S. Provisional Patent Application Ser. No. 63/724,048, filed Nov. 22, 2024, U.S. Provisional Patent Application Ser. No. 63/724,707, filed Nov. 25, 2024, and U.S. Provisional Patent Application Ser. No. 63/767,817, filed Mar. 6, 2025. The disclosures of each of which are incorporated herein by reference in their entirety.

### FIELD OF THE INVENTION

The present invention relates to certain zwitterionic metal chelators and their use as imaging, diagnostic, chemical processing, and treatment agents. The zwitterionic metal chelators described herein, have desirable properties that maximize solubility in aqueous environments, minimize non-specific interactions, and retain the ability to target thus resulting in an improved performance in a variety of medical, agricultural, and chemical processes. In in vivo and medical applications, zwitterionic metal chelators improve the signal-to-background ratio and therapeutic window as compared to other metal chelators while retaining high stability.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 18, 2025, is entitled “1515138_116WO2_SL.xml”, and is 6,043 bytes in size.

### BACKGROUND OF THE INVENTION

Cationic metals are insoluble in water. For this reason, whenever they are needed in a medical, agricultural, or chemical process they must be bound to, e.g. by coordination or chelation, to an organic compound that renders the complex soluble in aqueous environments and less toxic. Previously described metal chelators pay little attention to the polyionicity and sphere of hydration that is required to fully isolate the metal and thus result in unwanted non-specific interactions.

Chelated metals are commonly used in a variety of current imaging techniques. These techniques, like magnetic resonance imaging (MRI), single-photon emission computed tomography (SPECT), or positron emission tomography (PET), permit the detection of human diseases and conditions.

However, in the applications mentioned above, the metal isotope needs to be bound to metal chelators to prevent toxic effects and, for targeted agents, to maintain association with the targeting vector. Many common metal chelators, such as DOTA, PyC3A, and macropa, lack the ability to be tuned with respect to the pharmacokinetics, solubility, non-specific tissue/organ uptake, and plasma-protein binding. In addition, common metal chelators are not easily cleared from the body resulting in accumulation in off-target tissues and organs. For diagnostic imaging, this results in higher background. For radiotherapy, this results in toxicity without benefit.

Similarly, in any agricultural or chemical processes that require metal chelation, little attention is currently paid to the level of aqueous solubility attained or mechanisms to minimize non-specific interactions that either lower the yield of the process or fail to block side reactions.

Some research has been undertaken to provide derivatives of these chelators by allowing for the conjugation of targeting vectors, such as antibodies, peptides, small molecules, and steroids. These derivatives are often produced by replacement of one or more carboxylic acid arms of the chelators to include a targeting vector. However, such replacement often has a substantial impact on the chelating properties of the chelator-rendering their metal-binding properties inferior, if not altogether useless for biomedical applications. In some instances, replacement alters the biodistribution and/or clearance of the molecule, which can lead to a higher background or a smaller therapeutic window.

As such, there remains a need for new and improved agents with high stability that maximize the solubility of metal complexes in aqueous environments, minimize non-specific/off-target interactions, equilibrate rapidly between the intravascular and extravascular spaces when injected into the body and are then cleared efficiently from the body, including by renal filtration. The zwitterionic metal chelators of the invention are directed toward these and other needs.

### SUMMARY OF THE INVENTION

This invention provides for zwitterionic metal chelators which are useful for various medical, agriculture, and chemical processes. These chelators provide improved properties such as high solubility in aqueous environments and low non-specific interactions. When used in medical applications they can increase the signal-to-background ratio of imaged tissues and the therapeutic window of treated tissue, while allowing for easier and more efficient clearance by the subject.

In one aspect, the disclosure provides a zwitterionic metal chelator complex comprising a metal chelator having one or more zwitterionic groups and a metal or metal isotope selected from the group consisting of a radionuclide, a label, a paramagnetic metal and a heavy metal.

In certain embodiments, the metal chelator is a derivative of 3,6,9,15-Tetraazabicyclo[9.3.1] pentadeca-1 (15), 11, 13-triene-3,6,9-triacetic acid (PCTA). In still other embodiments, the zwitterionic metal chelator complexes further comprise one or more targeting vectors. In particular embodiments, the one or more targeting vectors are cRGD, PSMA-617, FAPI, octreotide, a bombesin analog, or a homo- or hetero-dimer formed from their combination.

In certain embodiments, wherein the metal chelator is a derivative of PCTA, the metal or metal isotope is Pb, Mn, Zr, Cu, Ga, In, Y, Gd, Lu, Ac, or Tb, or Gd or Gd³⁺. In certain embodiments, the metal chelator is a derivative of PCTA, the metal or metal isotope is Gd or Gd³⁺. In some embodiments, the zwitterionic metal chelator of the zwitterionic metal chelator complex has the formula:

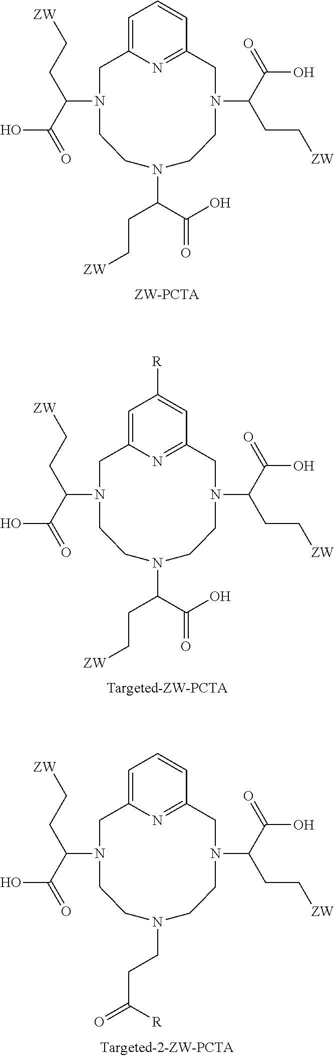

wherein ZW represents a zwitterionic group. In certain embodiments, ZW represents an ammonium sulfobetaine group. In certain embodiments, ZW represents an N-oxide group. Substituents R can include a targeting vector or a reactive group.

In certain embodiments, the zwitterionic metal chelator complexes further comprise one or more targeting vectors. In particular embodiments, the one or more targeting vectors are cRGD, PSMA-617, FAPI, octreotide, a bombesin analog, or a homo- or hetero-dimer formed from their combination.

In particular, these chelators allow for lower temperature, and even ambient temperature, chelation of metals. This fast, room temperature chelation allows for more efficient attachment to proteins including, but not limited to monoclonal antibodies which is not achievable with previously known chelators, such as DOTA.

In certain embodiments, the metal chelator is an amide derivative of PCTA and the metal or metal isotope is Gd or Gd³⁺. In certain embodiments, the metal chelator is an amide derivative of PCTA and the metal or metal isotope is Pb, Mn, Pd, Zr, Cu, Ga, In, Y, Lu, Ac, or Tb.

In some embodiments, the zwitterionic metal chelator of the zwitterionic metal chelator complex has the formula:

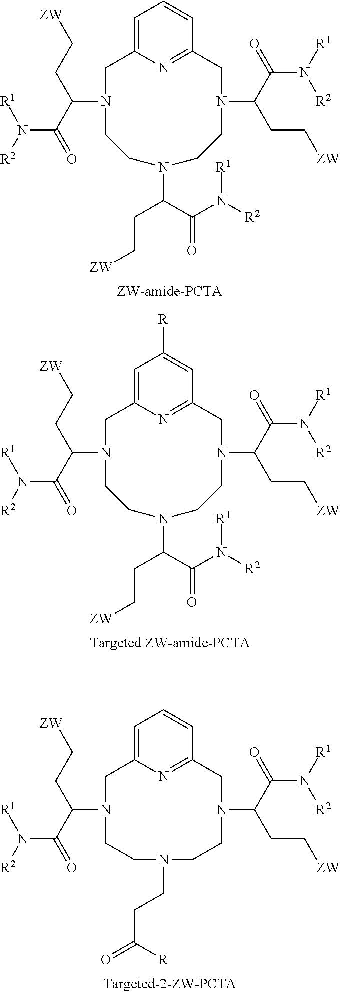

wherein ZW represents a zwitterionic group. In certain embodiments, ZW represents an ammonium sulfobetaine group. In certain embodiments, ZW represents an N-oxide group. Substituents R can be a targeting vector or a reactive group. Substituents R¹ and R² can be the same or different and are H, alkyl or aryl groups. In certain embodiments, substituents R¹ and R² are each H.

In certain embodiments, the zwitterionic metal chelator complexes further comprise one or more targeting vectors. In particular embodiments, the one or more targeting vectors are cRGD, PSMA-617, FAPI, octreotide, a bombesin analog, or a homo- or hetero-dimer formed from their combination.

In certain embodiments, the metal chelator is a derivative of PCTP. In still other embodiments, the zwitterionic metal chelator complexes further comprise one or more targeting vectors. In particular embodiments, the one or more targeting vectors are cRGD, PSMA-617, FAPI, octreotide, bombesin, or a homo- or hetero-dimer formed from their combination.

In some embodiments, wherein the metal chelator is a derivative of PCTP, the metal or metal isotope is Pb, Mn, Pd, Zr, Cu, Ga, In, Y, Gd, Lu, Ac, or Tb.

In other embodiments, wherein the metal chelator is PCTP, the metal or metal isotope is Pb.

In some embodiments, the zwitterionic metal chelator of the zwitterionic metal chelator complex has the formula:

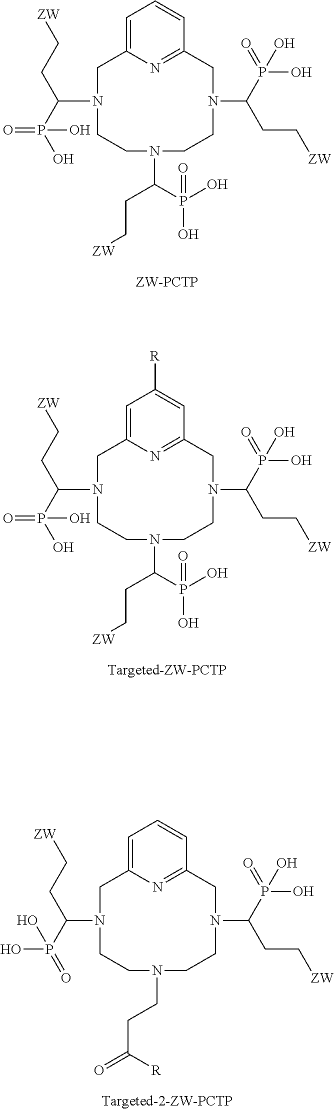

in which ZW represents a zwitterionic group and substituents. R can be a targeting vector or a reactive group.

In certain embodiments, ZW represents an ammonium sulfobetaine group. In certain embodiments, ZW represents an N-oxide group.

In another aspect, the disclosure provides an imaging agent comprising a zwitterionic metal chelator complex according to the disclosure.

In another aspect, the disclosure provides a method of imaging cells, tissues, or organs, the method comprising:
(a) contacting cells with an imaging agent according to the disclosure; and
(b) imaging the cells, tissues, or organs using positron emission tomography (PET), single-photon emission computerized tomography (SPECT), or magnetic resonance imaging (MRI).

In some embodiments of the method of imaging, the cells are tumor cells or cells undergoing angiogenesis.

In other embodiments of the method of imaging, t the imaging agent is administered to an organism comprising or suspected of comprising the cells.

In particular embodiments of the method of imaging, the organism is human.

In some embodiments of the method of imaging, the tissue or cells is imaged in vivo.

In another aspect, the disclosure provides therapeutic agent comprising a zwitterionic metal chelator complex according to the disclosure and a pharmaceutically acceptable carrier or excipient.

In another aspect, the disclosure provides a method of treating a cancerous condition in a subject in need thereof, the method comprising:
contacting cancer cells in the subject with an effective amount of a therapeutic agent according to the disclosure,
wherein the metal atom complexed to the zwitterionic metal chelator is:
   a radioactive metal isotope known to emit ionizing radiation that results in the death of cells that take up the analogs;
   or a non-radioactive metal that is capable of releasing cytotoxic radiation upon irradiation with alpha emission, beta emission, neutron capture, or a combination thereof.

In some embodiments of the method of treatment, the metal atom complexed to the zwitterionic metal chelator is a non-radioactive metal that is capable of releasing cytotoxic radiation upon irradiation with alpha emission, beta emission, neutron capture, or a combination thereof; the method further comprising a step of irradiating the tumor cells using alpha emission, beta emission, neutron capture, or a combination thereof.

In some other embodiments of the method of treatment, the cancer cells are adult solid tumor cells or pediatric solid tumor cells.

In other embodiments of the method of treatment, the cancer cells are melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In some embodiments of the method of treatment, the cancer cells are prostate cancer cells. In some other embodiments of the method of treatment, the cancer cells are malignant cancer cells.

In another aspect, the disclosure provides method of treating a non-cancerous condition in a subject in need thereof, the method comprising:
administering to the subject an effective amount of a therapeutic agent according to the disclosure,
wherein the metal atom complexed to the zwitterionic metal chelator is:
   a radioactive metal isotope known to emit ionizing radiation that results in a therapeutic effect on the subject.
   or a non-radioactive metal that is capable of releasing therapeutic radiation upon irradiation with alpha emission, beta emission, neutron capture, or a combination thereof.

In some embodiments of the method of treatment, the non-cancerous condition is a musculoskeletal disorders or a tissue hypertrophy disorder.

In some embodiments of the method of treatment, the subject is a human.

In another aspect, the disclosure provides a diagnostic agent comprising a zwitterionic metal chelator complex according to the disclosure and a pharmaceutically acceptable carrier or excipient.

In another aspect, the disclosure provides a method of measuring the efficacy of a biological system of a subject, the method comprising:
(a) administering a quantifiable amount of a diagnostic agent according to the disclosure to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In some embodiments of the method of measuring the efficacy of a biological system, the biological system is the renal system, the hepatic system, or the blood pool.

In another aspect, the disclosure provides a method of measuring the efficacy of renal function a subject, the method comprising:
(a) administering a quantifiable amount of a diagnostic agent according to the disclosure to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In another aspect, the disclosure provides a method of quantifying the glomerual filtration rate of a subject, the method comprising:
(a) administering a quantifiable amount of a diagnostic agent according to the disclosure to the subject;
(b) determining the amount of diagnostic agent present in the blood and urine of the subject as a function of time using either measurements of each bodily fluid or imaging the subject with positron emission tomography (PET), single-photon emission computerized tomography (SPECT), or magnetic resonance imaging (MRI)

In another aspect, the disclosure provides method for removing toxic or excess metals in a subject in need of such treatment, the method comprising:
(a) administering a therapeutically effective amount of a therapeutic agent according to the disclosure to the subject,
wherein the therapeutic agent comprises a metal chelator having one or more zwitterionic groups which is not coordinated to a metal or metal isotope, and a pharmaceutically acceptable carrier or excipient.

In other embodiments of the method of removing toxic or excess metals in a subject, the toxic or excess metals are gadolinium.

In another aspect, the disclosure provides a radiosurgical method for treating a patient body, the method comprising:
receiving a desired lesion pattern and planned radiation distribution;
   administering an effective amount of a diagnostic agent according to the disclosure to the subject to effectively image the desired lesion pattern;
performing surgery on the desired lesion pattern to treat the patient body.

In some embodiments of the radiosurgical method of the disclosure, the zwitterionic metal chelator of the diagnostic agent further comprises one or more targeting vectors wherein the one or more targeting vectors are cRGD, PSMA-617, FAPI, octreotide, a bombesin analog, or a homo- or hetero-dimer formed from their combination.

In other embodiments of the radiosurgical method of the disclosure, the desired lesion pattern is received from a user interface of a treatment planning module.

In still other embodiments of the radiosurgical method of the disclosure, the treatment planning module is pre-programmed with specifications for various disease states and cancerous conditions.

In yet other embodiments of the radiosurgical method of the disclosure, the treatment planning module identifies lesion patterns for various disease states and cancerous conditions using artificial intelligence data.

In particular embodiments of the radiosurgical method of the disclosure, the n the surgery is performed using a sterotactic radiosurgical system.

In another aspect, the disclosure provides a method of treating a cancer by administering an effective amount of a therapeutic agent according to the disclosure, wherein the method comprises a step to diagnose the cancer and a step of administering the therapeutic agent to a subject determined to be in need thereof;
wherein the step to diagnose the cancer comprises:
   contacting cells, tissues or organs of a subject with an imaging agent,
   imaging the cells, tissues, or organs of the subject using positron emission tomography (PET), single-photon emission computerized tomography (SPECT), or magnetic resonance imaging (MRI), and
   diagnosing the cancer in the cells tissues, or organs of the subject based on imaging data collected;
and wherein the metal atom complexed to the zwitterionic metal chelator is:
   a radioactive metal isotope known to emit ionizing radiation that results in the death of cells that take up the analogs;
   or a non-radioactive metal that is capable of releasing cytotoxic radiation upon irradiation with alpha emission, beta emission, neutron capture, or a combination thereof.

### BRIEF DESCRIPTION OF THE DRAWINGS

FIG. 1 is a representation of four zwitterionic metal chelators according to the invention.

FIG. 2 is a representation of Zwitterionic groups according to the invention.

FIG. 3 is a representation of three targeting vectors encompassed by the invention.

FIG. 4 is a representation of synthesis of the azide-functionalised chelator (R,R,R)-Bn₃-PCTAZA and its subsequent conversion into the zwitterionic chelators, followed by complexation with Gd(III) and Eu(III).

FIGS. 5A, 5B, and 5C show stability data for the zwitterionic metal chelators according to the invention. FIG. 5A shows the release of free gadolinium over time for the Gd—SB₃-PCTA 11a, Gd-NOx₃-PCTA 12a, gadopiclenol 3 and Gd-DOTA 2 under acidic conditions (pH 1.2) at 37° C., as determined by complexometric titration with arsenazo III. FIG. 5B shows time-dependent relative longitudinal relaxation time T₁(t)/T₁(t₀) of gadolinium complexes (1.25 mM) in presence of ZnCl₂ (1.25 mM) in phosphate-buffered saline (PBS, pH 7.4, 37° C.), measured at 1.4 T. Measurements were performed at 0 h, 3 h, 1 day, 3 days, 7 days and 15 days. FIG. 5C shows stability of Gd(III) complexes in human serum at 37° C. Measurements were performed at 0 days, 1 day, 3 days, 7 days and 14 days.

FIGS. 6A, 6B, 6C, and 6D show relaxivity data for the zwitterionic metal chelators according to the invention. FIG. 6A shows longitudinal relaxivity (r₁) determined from plotting the reciprocal relaxation time 1/T₁ versus the concentration of Gd—SB₃-PCTA 11a, Gd-NOx₃-PCTA 12a and gadopiclenol 3 in H₂O at 37° C. and 1.4 T (60 MHz). FIG. 6B shows Longitudinal relaxivity (r₁) determined from plotting the reciprocal relaxation time 1/T₁ versus the concentration of Gd—SB₃-PCTA 11a, Gd-NOx₃-PCTA 12a and gadopiclenol 3 in H₂O at room temperature and 7 T (300 MHz) FIG. 6C shows representative MR images acquired at 7 T at multiple inversion times (TI) ranging from 50 ms to 3200 ms of Gd—SB₃-PCTA 11a, Gd-NOx₃-PCTA 12a and gadopiclenol 3 in a phantom holder and surrounded by water. Lighter circles represent the regions of interest used for subsequent quantitative analysis. FIG. 6D shows a R₁ relaxation rate map acquired at 7 T with an inversion-recovery spin-echo sequence with multiple inversion time delays.

FIG. 7 depicts a solid-phase synthetic scheme (Scheme 2) which produces an azide functionality to which the zwitterinic groups can be added using click-chemistry methods as described herein and as would be familiar to one of the art.

FIG. 8 depicts examples of click-chemistry linked zwitterionic groups on PCTA-based zwitterionic metal chelators of the invention. FIG. 8 also these compares PCTA-based zwitterionic metal chelators of the invention to other chelating materials-gadapiclenol and DOTA. In each of the examples of FIG. 8, Gd is the metal in the chelation complex.

### DETAILED DESCRIPTION

The present disclosure relates, inter alia, to an imaging, diagnostic, therapeutic or chemical agent that is composed of a zwitterionic metal chelator. In some aspects, the agents described herein are useful in, for example, the detection of abnormal or diseased biological tissues and cells. In some aspects, the zwitterionic metal chelators are particularly useful for imaging whole organisms as they have improved in vivo behavior, such as low non-specific binding to non-targeted tissues and high stability, resulting in an improved signal-to-background ratio in connection with the detected signal and/or improved therapeutic window. Similarly, in an agricultural or chemical process that now uses a metal chelator, zwitterionic metal chelators will improve solubility and minimize non-specific interactions. It is believed that these improved properties result from the balancing of formal charges on the metal chelator, rendering a polyionic yet “charge-balanced” molecule having a net charge that is neutral or close to neutral, with an extended sphere of hydration and better isolation of the metal.

### DEFINITIONS AND ADDITIONAL EMBODIMENTS

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a zwitterionic metal chelator of the invention and/or prodrugs thereof to a subject in need of diagnosis or treatment. As used herein, the term “ligand”, “targeting vector”, or “targeting ligand” refers to a moiety which is bound to or coordinated to the therapeutic/imaging agents or zwitterionic metal chelators of the composition of the invention to provide enhanced binding to particular cell types or an increased concentration in the presence of particular cell types. In certain embodiments, the targeting vector can be bound to the therapeutic/imaging agents or zwitterionic metal chelators of the compositions in addition to the zwitterionic groups thereon. In still other embodiments, the targeting vector can be bound to the zwitterionic metal chelator in place of one or more zwitterionic groups provided that the zwitterionic metal chelator retains at least one zwitterionic group.

As used herein, the term “therapeutic window” or “therapeutic index” refers to the relationship between the therapeutic and toxic dose of a given drug and is calculated using the ED50 and TD50 (Therapeutic Index=TD50/ED50). In certain embodiments of the invention, the zwitterionic metal chelators of the invention have a higher therapeutic index relative to other metal chelators. In certain other embodiments, the therapeutic window refers to a certainty safety factor (CSF) which is defined herein as the ratio of [TD1/ED99]. A CSF>1 indicates that the dose effective in 99% of the population is less than the dose that would be toxic in 1% of the population. In certain embodiments of the invention, the zwitterionic metal chelators of the invention have a higher CSF relative to other metal chelators.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

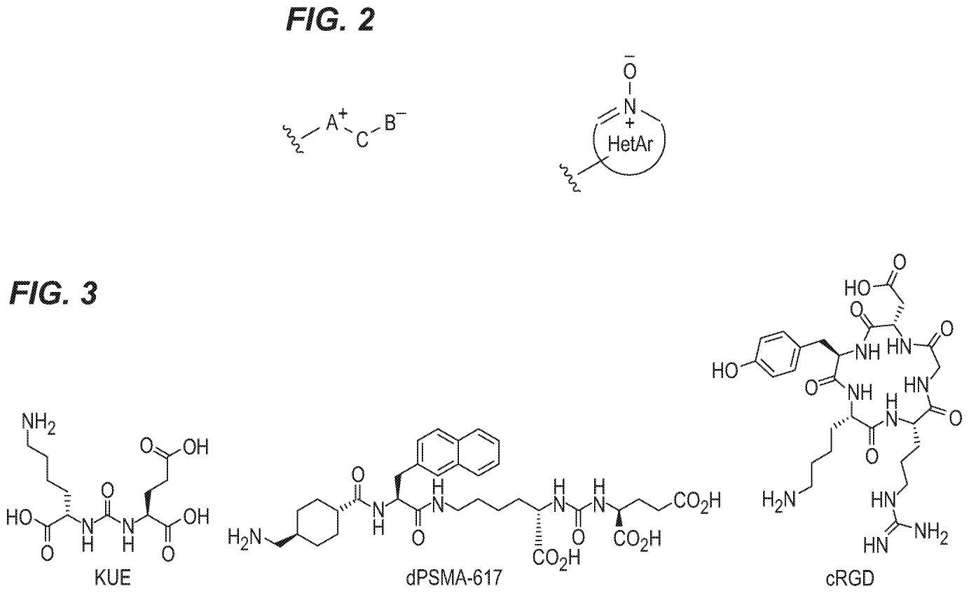

As used herein, the term “zwitterionic group,” “zwitterionic ligand,” or “zwitterion” refer to one or more charged moieties or ligands which are present on or can be reacted with a metal chelator core. The zwitterionic metal chelators of the claimed invention are decorated by one or more zwitterionic groups, i.e., moieties combining positive (A+, FIG. 3) and negative charges (B−, FIG. 2) that sum to a net charge of zero. These zwitterionic groups are distinct from the chelator core itself, which often has negative charges to chelate positively-charged metals. For example, a zwitterionic metal chelator with a chelator core of −4 that binds a +4 metal would have a total net charge of zero. Although a total net charge of zero is considered ideal, a zwitterionic metal chelator with a chelator core of −4 that binds a +2 metal, resulting in an overall charge of −2, would still be expected to exhibit improved properties in vivo because of shielding of the chelator core/metal complex by one or more zwitterionic groups. In the absence of zwitterionic groups, the molecule would have no such shielding or expanded water of hydration and would be more likely to bind non-specifically.

A particular active agent molecule may have several attached “zwitterionic groups” or charge pairs (also described herein as “ZW group(s)”). In general, the anion portion and the cation portion of the zwitterionic group (charge pair) will be part of the same moiety, though it is possible for two ionic groups to be used as separate moieties to form a zwitterionic group. In particular embodiments, the zwitterionic group is covalently bound to the base structure via a carbon-carbon bond, a carbon-oxygen bond, or a nitrogen-carbon bond. Examples of zwitterionic groups (charge pairs) that can be included in the compounds and complexes of the claimed invention include, but are not limited to, ammoniophosphates, ammoniophosphonates, ammoniophosphinates, ammoniosulfonates, ammoniosulfates, ammoniocarboxylates, ammoniosulfonamides, ammonio-sulfon-imides, guanidiniocarboxylates, pyridiniocarboxylates, pyridiniosulfonates, ammonio (alkoxy)dicyanoethenolates, ammonioboronates, sulfoniocarboxylates, phophoniosulfonates, and phosphoniocarboxylates. The charged groups in these zwitterions can be separated by suitable spacer groups (C in FIG. 2) like linear or branched alkyl chains, aryl or heteroaryl moieties. In certain embodiments, the zwitterionic groups can be derivatives of amino acids, such as amino carboxylic acids, amino phosphonic acids, amino phosphinic acids or amino sulfonic acids, furthermore, aminoalkyl substituted sulfates or phosphates. Zwitterions can also be derivatives of betaines, such as carboxybetaines, sulfobetaines, sulfabetaines, phosphobetaines or phosphabetaines or N-oxides or derivatives of sulfamic acid. Particular examples of zwitterionic groups include ammonium sulfobetaines or N-oxides. A simple example of a zwitterionic group at physiological pH is the charge pair of a carboxylic acid (deprotonated at physiological pH) and an amine (protonated at physiological pH).

In some embodiments, the zwitterionic metal chelators of the invention can also comprise a targeting vector for an agricultural process, chemical process, disease, or tissue-specific epitope, such as the cyclic peptide cRGDyK (aka cRGD, FIG. 3) bound to one or more arms of the metal chelator. cRGD is a cyclic derivative of the tripeptide Arg-Gly-Asp which can be conjugated to one or more arms of the metal chelators of the invention. In still other embodiments, the targeting vector is octreotide or a bombesin analog. In other embodiments, the targeting vector is KUE or dPSMA-617, a GPI-derivative, a small molecule capable of targeting Fibroblast Activation Protein (FAP) also called FAP-inhibitor or FAPI, an amino acid or combination of amino acids, or derivatives thereof. In such embodiments, the targeting vector-conjugates can be formed in place of one or more zwitterionic groups. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

An ideal zwitterionic metal chelator conjugated to a targeting vector would adopt the total net charge of the targeting vector, which is purposeful because in most cases the charges on the targeting vector are crucial for the ability to bind its target. Targeted zwitterionic metal chelators thus retain the major advantage of minimizing non-specific binding while maximizing specific binding. It should be apparent to those skilled in the art that additional charges can be added to the zwitterionic metal chelator, if needed, to balance overall surface charge to zero.

In certain embodiments, the zwitterionic metal chelators of the invention chelator comprise a reactive linking group. Such reactive linking groups are typically an activated derivative of a carboxylic acid, such as an N-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alkohols, phenols, carbonyls, phosphanes, alkenes and tetrazines.

As used herein, the term “contacting” refers to the bringing together of substances in physical contact such that the substances can interact with each other. For example, when an agent is “contacted” with tissue or cells, the tissue or cells can interact with the agent, for example, allowing the possibility of binding interactions between the agent and molecular components of the tissue or cells. “Contacting” is meant to include the administration of a substance such as an agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO₃¹⁻), oxide, sulfinate, carboxylate, phosphinate, phosphonate, phosphate, and esters (such as alkyl esters) thereof. In some embodiments, the charged substituent is sulfonate or oxide. Examples of charged cationic substituents include quaternary ammonium ions (—NR₃⁺) and phosphonium ions (—PR₃⁺), where R is independently selected from C₁₋₆ linear alkyl, C₄₋₆ branched alkyl, C₃₋₆ cycloalkyl, aryl, heteroaryl and arylalkyl or heteroarylalkyl. Other charged cationic substituents include protonated primary, secondary, and tertiary amines, as well as guanidinium or amidinium or pyridinium or other protonated, alkylated or oxygenated nitrogen heterocycles. In some embodiments, the charged substituent is —N(CH₃)₃⁺.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, a peptide and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, an urea group, an alcohol group, an ether group, a sulfide group, an amino group, an alkylene group, an alkyne group, an azide group, a tetrazine, an amide group, a carbonyl group, or a phosphate group.

Some examples of solubilizing groups include polyethylene glycols, such as —(CH₂CH₂O)ₐ—H, —OC(═O)O(CH₂CH₂O)ₐH, —OC(═O)O(CH₂CH₂O)ₐCH₃, —O(CH₂CH₂O)ₐCH₃, and —S(CH₂CH₂O)₂CH₃, “a” being an integer between about 2 and about 25O. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH₂)_{c}— (OCH₂CH₂)_{d}—OR^{a}, wherein “c” is 0 to 6, “d” is 1 to 200, and R^{a} is H or C₁₋₆ alkyl. In some embodiments, “c” is 1 to 4, “d” is 1 to 10, and R^{a} is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

As used herein, the term “azido derivative(s)” refers to chemical compounds that contain one or more azido groups (—N₃) attached to their molecular structure. In certain embodiments, the azido group is a functional group consisting of three nitrogen atoms (N) connected in a linear arrangement, typically represented as —N₃. In certain embodiments, the azido derivatives include one or more of Fmoc-Azidoalanine, Fmoc-Azidohomoalanine, Fmoc-Azidophenylalanine, Fmoc-Azidoglycine, Fmoc-Azidoornithine, Fmoc-Azidolysine, Fmoc-Azidoaspartic Acid.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −1 are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a −1 charge. Suitable counterions with −1 charge include F—, Cl—, Br—, I—, sulfate, phosphate, perchlorate, acetate, trifluoroacetate, maleate, fumarate, mesylate, lactate, pyruvate, laevulinate, gluconate and the like.

In certain aspects, the disclosure is directed to zwitterionic metal chelators coordinated to or labeled with a metal or metal isotope. In certain embodiments, the zwitterionic metal chelators coordinated to or labeled with a metal or metal isotope can be used medical, agricultural, or chemical processing applications.

Exemplary medical applications include applications for detection, imaging, or treatment in/of a subject or in a biological sample In certain embodiments, the disclosure further provides for treatment of benign and malignant tumors and tumor cells using a zwitterionic metal chelators labeled with a radioactive metal isotope known to emit ionizing radiation in a form that would result in the death of cells that take up the analogs labeled with the radioactive metal isotope. Metal chelators labeled with certain non-radioactive metal isotopes, such as Zr or Gd, can be used for neutron capture therapy.

Various zwitterionic metal chelators of the invention, particularly chelators suitable for use in the imaging methods provided by the invention, include one or more metals or radioisotopes capable of emitting one or more forms of radiation or other contrast (for example, effects on water relaxation) which are suitable for detection with any standard radiology method such as PET, SPECT, gamma cameras, MRI and the like.

For the disclosed methods of detecting/imaging benign or malignant tissues, any metal isotope known to emit radiation in a form that is readily detectable by conventional imaging means can be incorporated into the targeting backbone. Non-limiting examples of “conventional imaging means” include gamma ray detection, PET scanning, SPECT scanning, and MRT scanning. Non-limiting examples of metals that may be complexed with the PCTA derivative chelators of the invention include Gd. Non-limiting examples of metals that may be complexed with the amide-PCTA derivative chelators of the invention include Gd, Mn, Cu, Co, Y, In, Ga, Zr, Tc, Eu, Tb, Ac, Lu and other lanthanide or actinide metals. In some embodiments, the metals can be radioactive metal isotopes. Non-limiting examples of radioactive metal isotopes that may be used include Ga- 66, Ga-67, Ga-68, Cu-64, Cu-67, Y-86, Co-55, Zr-89, Sr-83, Mn-52, As-72, Sc-44, Gd-153, Co-57, In-111, Ac-225, Tb-152, Tb-155 or Tc-99m.

For the disclosed methods of therapeutically treating malignant tumors, any radioactive metal isotope known to emit ionizing radiation in a form that would result in the death of cells that take up the analogs labeled with the radioactive metal isotope can be incorporated by chelation in the zwitterionic metal chelators of the invention. So, too, can non-radioactive metals that are capable of capturing neutrons and releasing cytotoxic radiation (neutron capture therapy). In some embodiments, the radioactive metal isotope emits its ionizing radiation in a form that minimizes damage to tissue outside of the cells that take up the labeled analogs. In certain embodiments, the invention provides compounds comprising one or more radioisotope suitable for use in radiation therapy. In certain embodiments, the zwitterionic metal chelators of the invention comprise at least one radioactive isotope of technetium, rhenium, gallium, indium, copper, yttrium, actinium, bismuth, samarium, dysprosium, holmium, terbium, or lutetium, including radioactive isotopes selected from Tc-99m, Tc-94m, Re-186, Re-188, Ga-68, Cu-64, Cu-67, Y-90, Y-86, Ac-225, Bi-213, In-111, Sm-153, Ho-166, Lu-177, Sc-43, Sc-44, Sc-47, Tb-149, Tb-152, Tb-155, Tb-161, and Dy-166. In other embodiments, non-limiting examples of metals used in neutron capture therapy include Zr-88 or Gd-157.

For the disclosed methods, the oxidation state of the metal coordinated to the chelator is not particularly limited. In general, the oxidation state can be adjusted based on the particular zwitterionic metal chelator used and the particular medical application used. In certain embodiments, the metal coordinated to the chelator has an oxidation state ranging from +1 to +5.

Various zwitterionic metal chelators of the invention are capable of generating at least a 2:1 target or signal to background ratio of radiation intensity, or more preferably about a 5:1, about a 10:1 or about a 15:1 ratio of radiation intensity between target and background.

Further, the zwitterionic metal chelators of the invention are excreted from tissues of the body quickly, and without significant non-specific uptake by off-target tissues and organs, to prevent prolonged exposure to the radiation of a radiolabeled compound or the toxic effects of non-radioactive metals administered to the patient. Typically zwitterionic metal chelators of the invention are eliminated from the body in less than about 24 hours. More preferably, compounds of the invention are eliminated from the body in less than about 16 hours, 12 hours, 8 hours, 6 hours, 4 hours, 2 hours, 90 minutes, or 60 minutes. Typically preferred compounds are eliminated in between about 60 minutes and about 120 minutes.

In some embodiments, the clearance of the zwitterionic metal chelators comprising the imaging agent or therapeutic agent is from the tumor, tissue, or organ in the subject. In some embodiments, there is more rapid clearance of the zwitterionic metal chelators comprising the imaging agent from the kidneys than from the tumor of the subject.

In some embodiments, the zwitterionic metal chelators are stable in vivo such that substantially all, e.g., more than about 50%, 60%, 70%, 80%, or more preferably 90% of the injected compound is not metabolized by the body prior to excretion. In other embodiments, the zwitterionic metal chelators are stable in vivo.

### PCTA—Based Zwitterionic Metal Chelators

In one aspect, the invention provides a zwitterionic metal chelator based on 2-[3,9-bis[1-carboxylato-4-(2,3-dihydroxypropylamino)-4-oxobutyl]-3,6,9,15-tetrazabicyclo[9.3.1]pentadeca-1 (15), 11,13-trien-6-yl]-5-(2,3-dihydroxypropylamino)-5-oxopentanoate; gadolinium (3+), also known as gadopiclenol. In particular, the metal chelator is a derivative of 3,6,9,15-Tetraazabicyclo[9.3.1]pentadeca-1 (15), 11,13-triene-3,6,9-triacetic acid (PCTA). Such zwitterionic metal chelators include the following:

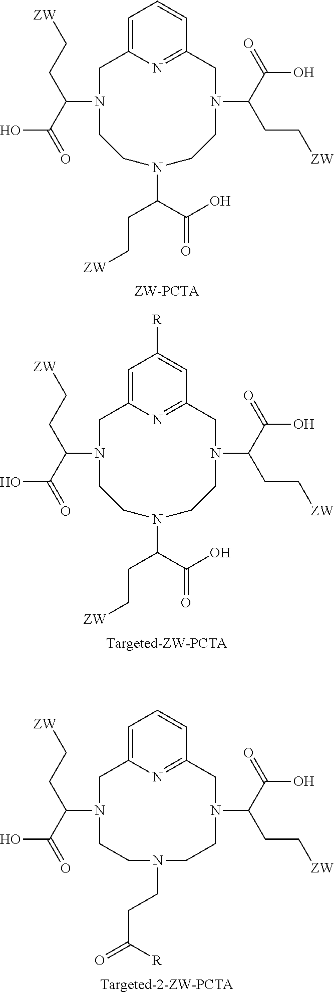

in which ZW represents a zwitterionic group. In certain embodiments, ZW represents an ammonium sulfobetaine group. In certain embodiments, ZW represents an N-oxide group. In certain embodiments, R represents a targeting vector. In other embodiments, R represents a carboxyl-substituted phenyl group. In still other embodiments, R represents a carboxyl-substituted phenyl group which forms an ester linkage to a targeting vector.

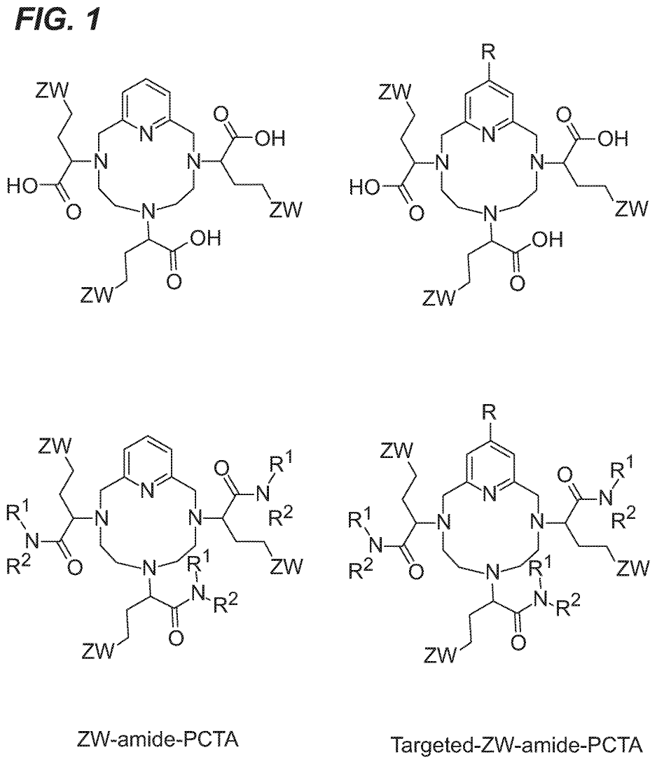

In such embodiments, a PCTA-based zwitterionic metal chelator contains stereogenic centers, any of these might have R or S-configuration. The PCTA-based zwitterionic metal chelator might be a single stereoisomer or might be a mixture of stereoisomers. The PCTA-based zwitterionic metal chelator includes 1, 2, 3, or 4 zwitterionic groups as defined in FIGS. 3 and 1, 2 or 3 reactive groups X for conjugation to targeting vectors or other molecules or materials. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In particular embodiments, the PCTA-based zwitterionic metal chelator includes 1, 2, 3, or 4 zwitterionic groups and 4 carboxylate groups for complexation to the metal or metal isotope. In still other embodiments, the PCTA-based zwitterionic metal chelator includes 1, 2, 3, or 4 zwitterionic groups, 4 carboxylate groups for complexation to the metal or metal isotope, and one or more targeting vectors. These targeting vectors can be bound to the chelator at any suitable structural location (e.g. reactive group X) as would be understood by one of ordinary skill in the synthesis of such compounds.

PCTA-based zwitterionic metal chelators can be synthesized using the protocols described in Scheme 1.

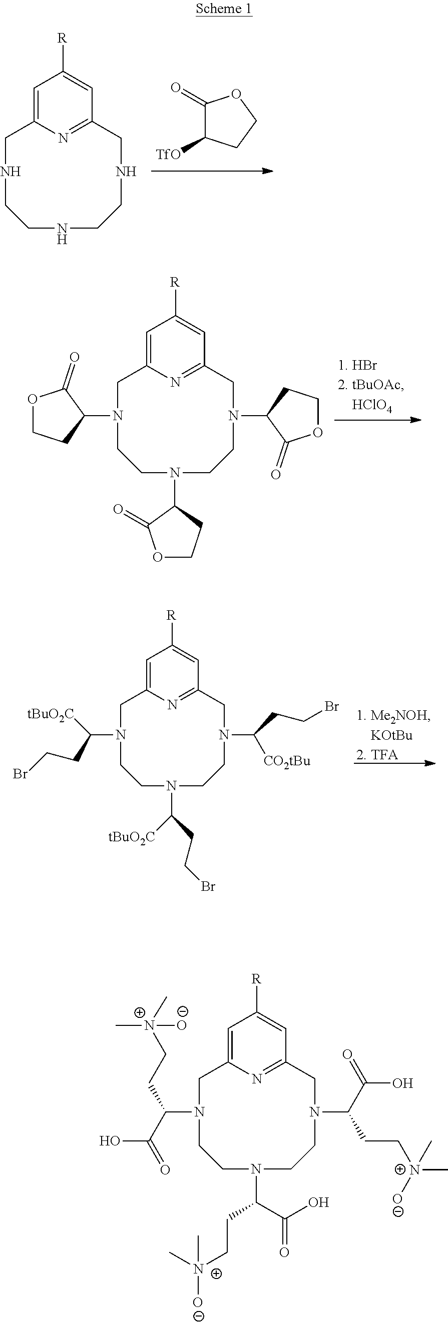

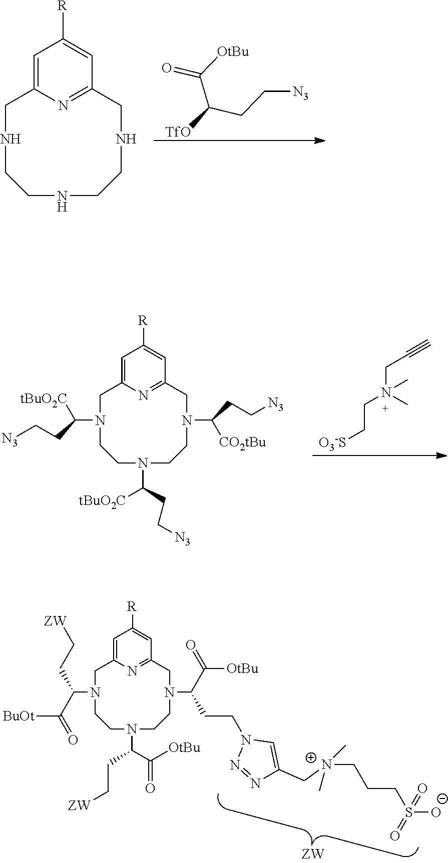

The protocols shown in the scheme provide an example for ZW to be an N-oxide group or a sulphobetaine group, optionally formed by click-chemistry (i.e. having a triazole ring).

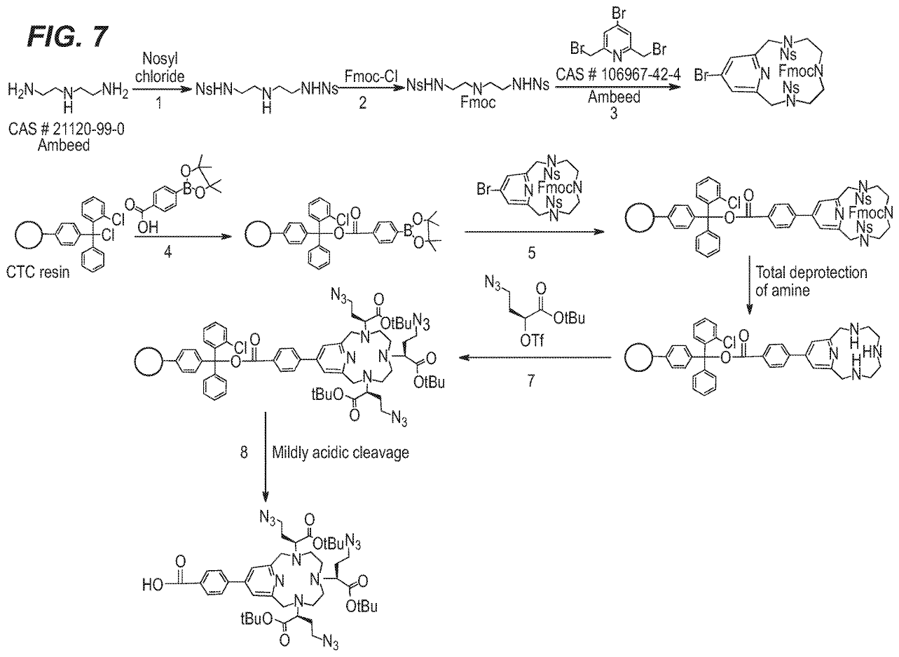

PCTA-based zwitterionic metal chelators can also be synthesized using a solid-phase protocol described in Scheme 2 (FIG. 7). Scheme 2 depicts a solid-phase synthetic scheme which produces an azide functionality to which the zwitterinic groups can be added using click-chemistry methods as described herein and as would be familiar to one of the art. Scheme 2 also permits conjugation to targeting ligands via the addition of a carboxylic acid.

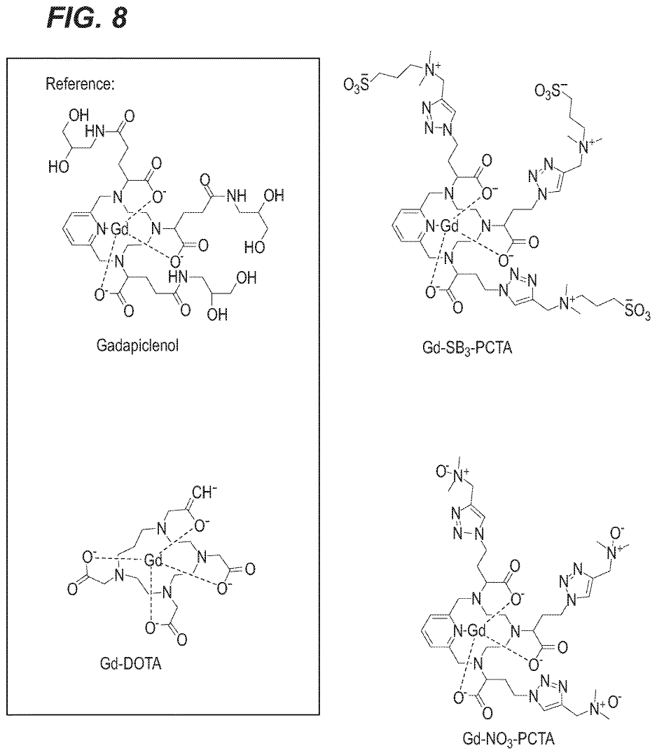

In Scheme 2, the PCTA-based zwitterionic metal chelator is formed using a solid-phase synthesis. In certain embodiments the solid phase material is a resin or coated glass bead, bed, or sheet which tethers the molecule during synthesis. In certain embodiments, the solid-phase material is a CTC resin. Other resins which can be used in this synthesis include PAM resins, Wang resins, HMPB/HMBA resins, DHP resins, Wienreb aminomethyl resins, SASRIN resins, HMPB/HMBA, Rink Amide resins, Sieber Amide resins, PAL resins, BHA resins, Merrifield resins, CHEMMATRIX resins, TENTAGEL resins, and SCAL (Safety-Catch) resins. This type of synthesis results in a compound having a free COOH that can be coupled to targeting vectors or linkers—said linkers optionally including other free COOH groups or other zwitterionic groups. This synthesis also results in azide groups which can be used to form zwitterionic groups using click-chemistry; for example, ZW groups having an N-oxide group or a sulphobetaine group bound to the PCTA ring through a triazole ring. This type of click-chemistry linking of the zwitterionic groups can also be applied to Scheme 1. Examples of click-chemistry linked zwitterionic groups can be seen in FIG. 8 which compares PCTA-based zwitterionic metal chelators of the invention to other chelating materials—gadapiclenol and DOTA. In each of the examples of FIG. 8, Gd is the metal in the chelation complex, but any of the metal centers described herein can be used for in the complex.

In either synthesis, N-oxide functionalities may also be synthesized by nucleophilic displacement of a suitable leaving group (such as a halogenide or a sulfonate) instead of the group R in the scheme above. The nucleophile in these conversions is an N,N-dialkylated hydroxylamine, which can be protected at the hydroxyl group.

In a particular embodiment, the PCTA-based zwitterionic metal chelators can be complexed with Zr, Cu, Ga, In, Y, Gd, Lu, Ac, Pb, Mn, Tb or other metals.

In still other embodiments, one or more zwitterionic groups of the PCTA-based zwitterionic metal chelator can be replaced with a targeting vector, such as cRGD, dPSMA-617, KUE, a GPI-derivative, a FAP-targeting small molecule, octreotide, a bombesin analog, or their corresponding homo- or hetero-dimers provided that PCTA-based zwitterionic metal chelator remains zwitterionic. In certain embodiments, the targeting vector includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Alternatively, the targeting vector can be covalently attached to a reactive linking group of the chelator compound of the invention through standard coupling procedures. For example, the carboxyl or activated carboxyl group of a reactive linking group can react with a nucleophilic functionality on the targeting vector, such as an amine or alcohol derivative, to form an amide or ester linkage. Additional details for the conjugation can be found in WO 2008/017074 and in Frangioni et al. Molecular Imaging, Vol. 1 (4), 354-364 (2002), each of which is incorporated herein by reference in its entirety.

It should be apparent to those skilled in the art, that if a targeting vector replaces a zwitterionic group, that zwitterionic group can be restored by adding it to a linker between the zwitterionic metal chelator and the targeting vector. The zwitterions ZW can be separated from the chelator core structure by appropriate spacer moieties including alkyl, aryl or heteroaryl groups. The targeting vectors (tv) can be separated from the chelator core structure by appropriate spacer moieties including alkyl, aryl, heteroaryl, ether, ester, amide, imine and oxime groups. The spacers can also contain one or more ether or amide bond or a combination of both and might contain zwitterionic groups added to the sidechains of the spacer moiety. In certain embodiments, the spacer moieties can include one or more polyethylene glycol (PEG) units, one or more units derived from a sugar moiety, units having one or more anionic groups. In certain embodiments, one or more linkers may be independently be absent.

In certain embodiments, the agent further comprises a PEG-moiety to alter the circulation time in blood. Such moiety can be bound to the conjugate at any suitable structural location as would be understood by one of ordinary skill in the synthesis of such compounds.

In certain embodiments, the conjugate of the PCTA-based zwitterionic metal chelator and the targeting vector has the following formula with KUE, dPSMA-617, cRGD, FAPI, octreotide, a GPI-derivative, or a bombesin analog as the targeting vector (tv):

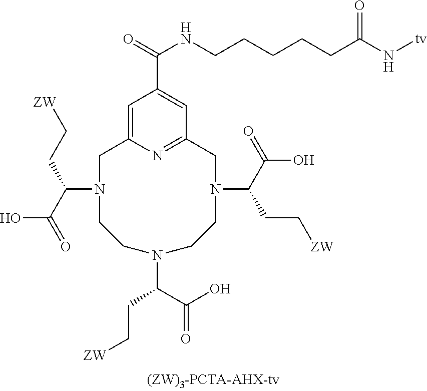

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In some cases, one of ordinary skill in the art would understand that one or more zwitterionic groups can be replaced with another moiety which could also impart hydrophilicity to the chelator. Such hydrophilicity-imparting groups may be substituted, added, or exchanged for alternative moieties that maintain or increase aqueous solubility and hydrophilicity of the molecule (e.g., a chelator or conjugate). Non-limiting examples of suitable hydrophilic groups include poly(ethylene glycol) (PEG) chains; saccharides and sugar derivatives (e.g., glucose, galactose, mannose, sialic acid); polyols and hydroxylated moieties (e.g., glycerol, sorbitol, mannitol, xylitol, diols, triols); carboxylic acids and carboxylates; sulfonic acids and sulfonates; sulfate groups; phosphate and phosphonate groups; amide, urea, and carbamate functionalities; amine oxides; sulfoxides; betaines (e.g., carboxybetaine, sulfobetaine); quaternary ammonium groups; guanidinium-containing groups; and polar heterocycles (e.g., morpholine, piperazine, N-vinylpyrrolidone). Such moieties may be introduced in protected or salt forms and can be tethered via suitable linkers to achieve the desired hydrophilicity, charge, and solubility characteristics.

### Amide-PCTA—Based Zwitterionic Metal Chelators

In one aspect, the invention provides a zwitterionic metal chelator based on Amide-PCTA. Amide-PCTA is a derivative of PCTA in which-COOH groups are replaced with —CONR₂ groups. Such zwitterionic metal chelators include the following:

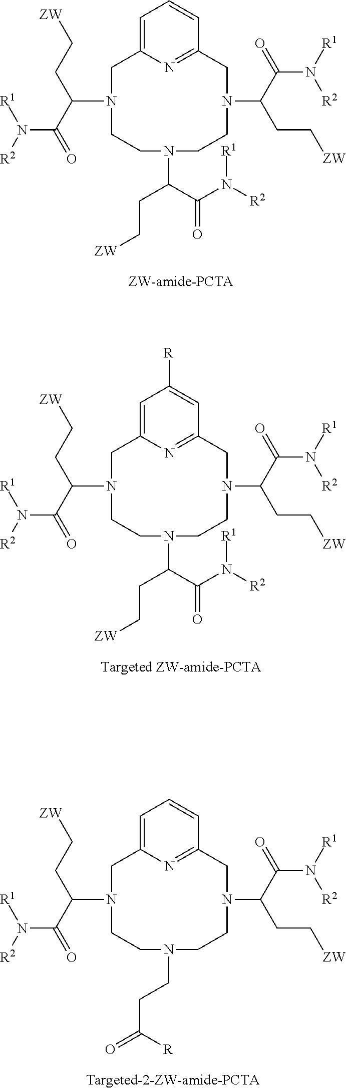

wherein ZW represents a zwitterionic group. Substituents R can be targeting vectors or reactive groups. Substituents R¹ and R² can be the same or different and are H, alkyl or aryl groups. In certain embodiments, substituents R¹ and R² are each H.

In certain embodiments, ZW represents an ammonium sulfobetaine group. In other embodiments ZW represents an N-oxide group.

In certain embodiments, R represents a targeting vector. In other embodiments, R represents a carboxyl-substituted phenyl group. In still other embodiments, R represents a carboxyl-substituted phenyl group which forms an ester linkage to a targeting vector.

In some embodiments, an amide-PCTA-based zwitterionic metal chelator contains stereogenic centers, any of these might have R or S-configuration. The amide-PCTA-based-based zwitterionic metal chelator might be a single stereoisomer or might be a mixture of stereoisomers. The amide-PCTA-based zwitterionic metal chelator includes 1, 2, 3, or 4 zwitterionic groups and 1, 2 or 3 reactive groups for conjugation to targeting vectors or other molecules or materials. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group can also be an amine, azide, alkyne, alkene, ketone, aldehyde, alcohol, phenol, maleimide, thiol, phosphane or a tetrazine. The zwitterionic groups ZW and the reactive groups can be separated from the chelator core structure by appropriate spacer moieties including alkyl, aryl or heteroaryl groups. In particular embodiments, the amide-PCTA-based zwitterionic metal chelator includes 1, 2, 3, or 4 zwitterionic groups and 4 carboxylate groups for complexation to the metal or metal isotope. In still other embodiments, the amide-PCTA-based zwitterionic metal chelator includes 1, 2, 3, or 4 zwitterionic groups, 4 carboxylate groups for complexation to the metal or metal isotope, and one or more targeting vectors. These targeting vectors can be bound to the chelator at any suitable structural location (e.g. at a reactive group) as would be understood by one of ordinary skill in the synthesis of such compounds.

Amide-PCTA-based zwitterionic metal chelators can be synthesized using the protocol described in Scheme 3.

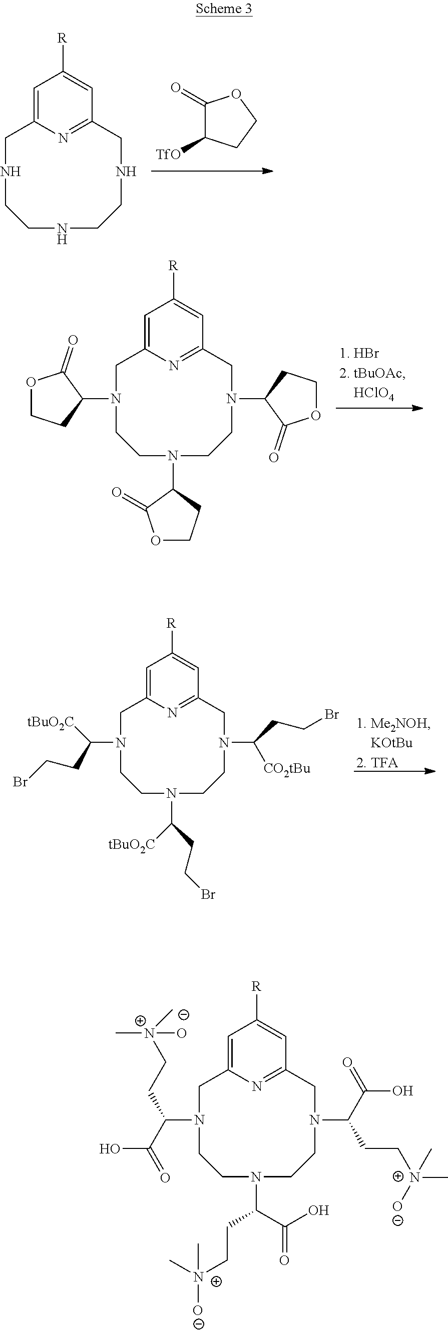

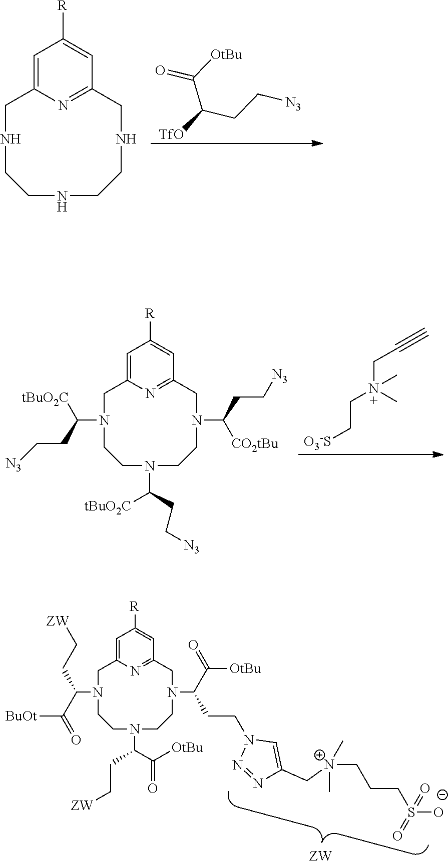

The protocols shown in the scheme provide an example for ZW to be an N-oxide group or a sulphobetaine group and R is a targeting vector or a carboxyl-substituted phenyl group.

The protocols of Scheme 1 (FIG. 7) may also be adapted to the amide-PCTA based zwitterionic metal chelators.

Similarly, in the preparation of amide-PCTA based zwitterionic metal chelators, click chemistry linkages can be used to prepare the zwitterionic functionality; for example, ZW groups having an N-oxide group or a sulphobetaine group bound to the PCTA derivative through a triazole ring.

In either synthesis, N-oxide functionalities may also be synthesized by nucleophilic displacement of a suitable leaving group (such as a halogenide or a sulfonate) instead of the group R in the scheme above. The nucleophile in these conversions is an N,N-dialkylated hydroxylamine, which can be protected at the hydroxyl group.

In certain embodiments, the amide-PCTA-based zwitterionic metal chelators can be complexed with Pb, Zr, Cu, Ga, In, Y, Gd, Lu, Ac, Tb, Mn, Pd or other metals.

In still other embodiments, one or more zwitterionic groups of the amide-PCTA-based zwitterionic metal chelator can be replaced with a targeting vector, such as cRGD, dPSMA-617, KUE, a GPI-derivative, a FAP-targeting small molecule, octreotide, a bombesin analog, or their corresponding homo- or hetero-dimers provided that amide-PCTA-based zwitterionic metal chelator remains zwitterionic. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Alternatively, the targeting vector can be covalently attached to a reactive linking group of the chelator compound of the invention through standard coupling procedures. For example, the carboxyl or activated carboxyl group of a reactive linking group can react with a nucleophilic functionality on the targeting vector, such as an amine or alcohol derivative, to form an amide or ester linkage. Additional details for the conjugation can be found in WO 2008/017074 and in Frangioni et al. Molecular Imaging, Vol. 1 (4), 354-364 (2002), each of which is incorporated herein by reference in its entirety.

It should be apparent to those skilled in the art, that if a targeting vector replaces a zwitterionic group, that zwitterionic group can be restored by adding it to the linker between the zwitterionic metal chelator and the targeting vector. In certain embodiments, the linker moieties can also include one or more polyethylene glycol (PEG) units, one or more units derived from a sugar moiety, units having one or more anionic groups.

In addition, the zwitterions can be separated from the chelator core structure by appropriate spacer moieties including alkyl, aryl or heteroaryl groups. The targeting vectors (tv) may also be separated from the chelator core structure by appropriate spacer moieties including alkyl, aryl, heteroaryl, ether, ester, amide, imine and oxime groups. The spacers can also contain one or more ether or amide bond or a combination of both and might contain zwitterionic groups added to the sidechains of the spacer moiety. In certain embodiments, one or more spacers may independently be absent.

In certain embodiments, the agent further comprises a PEG-moiety to alter the circulation time in blood. Such moiety can be bound to the conjugate at any suitable structural location as would be understood by one of ordinary skill in the synthesis of such compounds.

In certain embodiments, the conjugate of the amide-PCTA-based zwitterionic metal chelator and the targeting vector has the following formula with KUE, dPSMA-617, cRGD, FAPI, octreotide, a GPI-derivative, a bombesin analog, or as the targeting vector (tv):

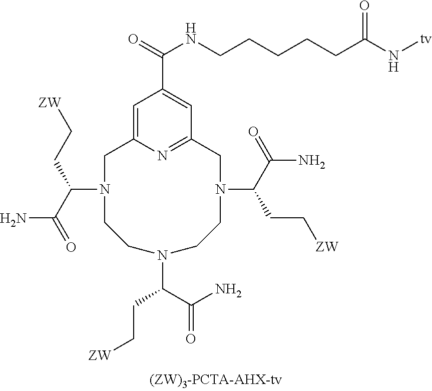

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In some cases, one of ordinary skill in the art would understand that one or more zwitterionic groups can be replaced with another moiety which could also impart hydrophilicity to the chelator. Such hydrophilicity-imparting groups may be substituted, added, or exchanged for alternative moieties that maintain or increase aqueous solubility and hydrophilicity of the molecule (e.g., a chelator or conjugate). Non-limiting examples of suitable hydrophilic groups include poly(ethylene glycol) (PEG) chains; saccharides and sugar derivatives (e.g., glucose, galactose, mannose, sialic acid); polyols and hydroxylated moieties (e.g., glycerol, sorbitol, mannitol, xylitol, diols, triols); carboxylic acids and carboxylates; sulfonic acids and sulfonates; sulfate groups; phosphate and phosphonate groups; amide, urea, and carbamate functionalities; amine oxides; sulfoxides; betaines (e.g., carboxybetaine, sulfobetaine); quaternary ammonium groups; guanidinium-containing groups; and polar heterocycles (e.g., morpholine, piperazine, N-vinylpyrrolidone). Such moieties may be introduced in protected or salt forms and can be tethered via suitable linkers to achieve the desired hydrophilicity, charge, and solubility characteristics.

### PCTP—Based Zwitterionic Metal Chelators

In one aspect, the invention provides a zwitterionic metal chelator based on 2-[3,9-bis[1-carboxylato-4-(2,3-dihydroxypropylamino)-4-oxobutyl]-3,6,9,15-tetrazabicyclo[9.3.1]pentadeca-1 (15), 11,13-trien-6-yl]-5-(2,3-dihydroxypropylamino)-5-oxopentanoate; gadolinium (3+), also known as gadopiclenol. In particular, the metal chelator is a derivative of 3,6,9,15-Tetraazabicyclo[9.3.1]pentadeca-1 (15), 11,13-triene-3,6,9-triacetic acid (PCTA) in which-COOH groups are replaced with —PO(OH)₂ groups. Such zwitterionic metal chelators include the following:

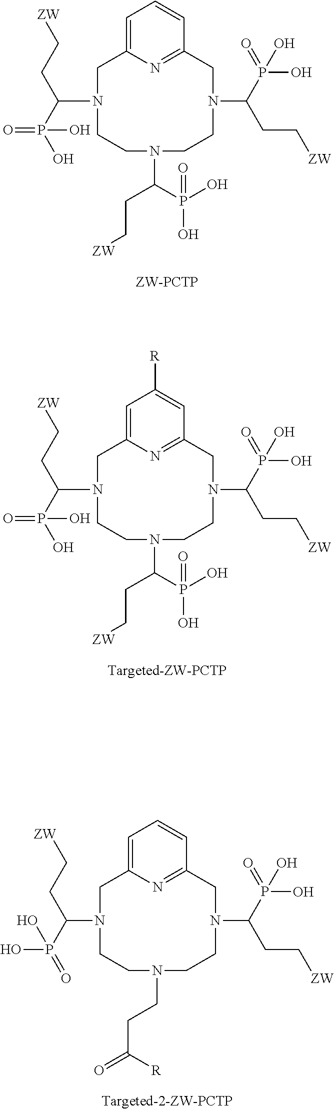

in which ZW represents a zwitterionic group. In certain embodiments, ZW represents an ammonium sulfobetaine group. In certain embodiments, ZW represents an N-oxide group and rR represents a targeting vector or reactive group. In certain embodiments, R represents a targeting vector. In other embodiments, R represents a carboxyl-substituted phenyl group. In still other embodiments, R represents a carboxyl-substituted phenyl group which forms an ester linkage to a targeting vector.

In such embodiments, a PCTA-based zwitterionic metal chelator contains stereogenic centers, any of these might have R or S-configuration. The PCTA-based zwitterionic metal chelator might be a single stereoisomer or might be a mixture of stereoisomers. The PCTA-based zwitterionic metal chelator includes 1, 2, 3, or 4 zwitterionic groups as defined in FIGS. 3 and 1, 2 or 3 reactive groups X for conjugation to targeting vectors or other molecules or materials. This reactive group can be a carboxylic acid, an activated derivative of a carboxylic acid, such as an NHS ester, a sulfo-NHS ester, a PFP ester, a HOBt ester, a HOAt ester, a TFP ester, an acid anhydride, an acid azide or an acid halide. The reactive group X can also be an amine, azide, alkyne, alkene, ketone, aldehyde, alcohol, phenol, maleimide, thiol, phosphane or a tetrazine. The zwitterionic groups ZWI and the reactive groups X can be separated from the chelator core structure by appropriate spacer moieties W and Y including alkyl, aryl or heteroaryl groups. In particular embodiments, the PCTA-based zwitterionic metal chelator includes 1, 2, 3, or 4 zwitterionic groups and 4 carboxylate groups for complexation to the metal or metal isotope. In still other embodiments, the PCTA-based zwitterionic metal chelator includes 1, 2, 3, or 4 zwitterionic groups, 4 carboxylate groups for complexation to the metal or metal isotope, and one or more targeting vectors. These targeting vectors can be bound to the chelator at any suitable structural location (e.g. reactive group X) as would be understood by one of ordinary skill in the synthesis of such compounds.

PCTP-based zwitterionic metal chelators can be synthesized using the protocols described in Scheme 4.

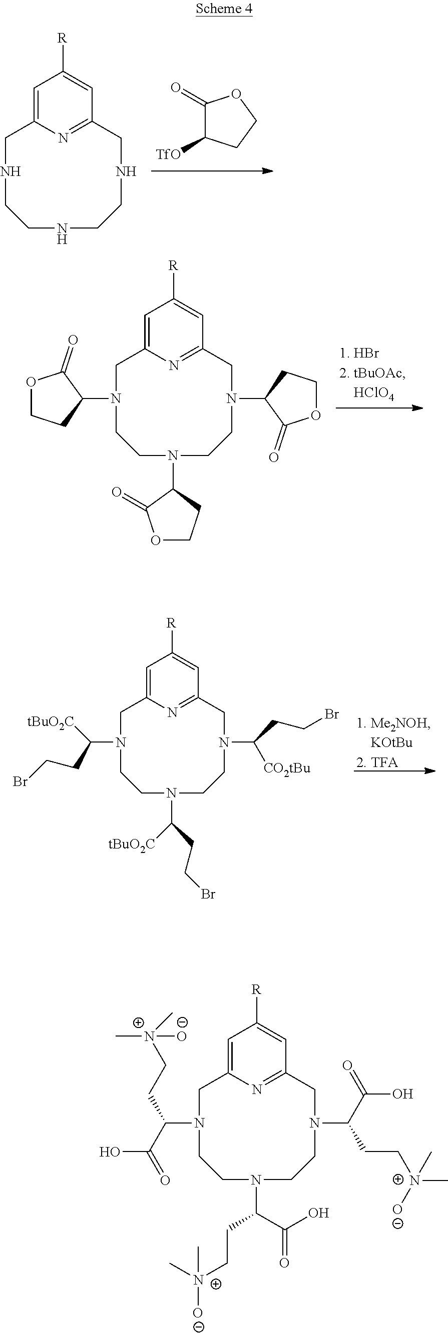

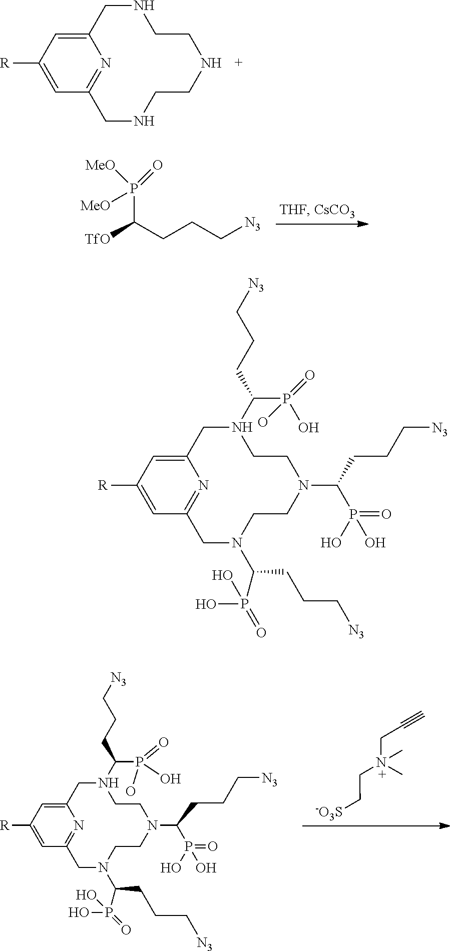

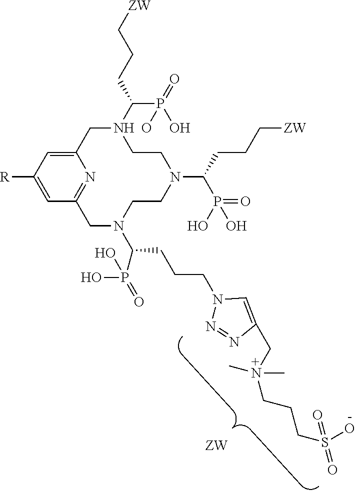

The protocols shown in the scheme can be modified to provide an example for ZW to be an N-oxide group or a sulphobetaine group.

The protocols of Scheme 1 (FIG. 7) may also be adapted to the PCTP based zwitterionic metal chelators.

Similarly, in the preparation of PCTP based zwitterionic metal chelators, click chemistry linkages can be used to prepare the zwitterionic functionality; for example, ZW groups having an N-oxide group or a sulphobetaine group bound to the PCTP ring through a triazole ring.

In either synthesis, N-oxide functionalities may also be synthesized by nucleophilic displacement of a suitable leaving group (such as a halogenide or a sulfonate) instead of the group R in the scheme above. The nucleophile in these conversions is an N,N-dialkylated hydroxylamine, which can be protected at the hydroxyl group.

In a particular embodiment, the PCTP-based zwitterionic metal chelators can be complexed with Zr, Cu, Ga, In, Y, Gd, Lu, Ac, Pb, Mn, Tb or other metals.

In still other embodiments, one or more zwitterionic groups of the PCTP-based zwitterionic metal chelator can be replaced with a targeting vector, such as cRGD, dPSMA-617, KUE, a FAP-targeting small molecule, octreotide, a bombesin analog, or their corresponding homo- or hetero-dimers provided that PCTA-based zwitterionic metal chelator remains zwitterionic. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Alternatively, the targeting vector can be covalently attached to a reactive linking group of the chelator compound of the invention through standard coupling procedures. For example, the carboxyl or activated carboxyl group of a reactive linking group can react with a nucleophilic functionality on the targeting vector, such as an amine or alcohol derivative, to form an amide or ester linkage. Additional details for the conjugation can be found in WO 2008/017074 and in Frangioni et al. Molecular Imaging, Vol. 1 (4), 354-364 (2002), each of which is incorporated herein by reference in its entirety.

It should be apparent to those skilled in the art, that if a targeting vector replaces a zwitterionic group, that zwitterionic group can be restored by adding it to a linker between the zwitterionic metal chelator and the targeting vector. The zwitterions ZW can be separated from the chelator core structure by appropriate spacer moieties including alkyl, aryl or heteroaryl groups. The targeting vectors (tv) can be separated from the chelator core structure by appropriate spacer moieties including alkyl, aryl, heteroaryl, ether, ester, amide, imine and oxime groups. The spacers can also contain one or more ether or amide bond or a combination of both and might contain zwitterionic groups added to the sidechains of the spacer moiety. In certain embodiments, the spacer moieties can include one or more polyethylene glycol (PEG) units, one or more units derived from a sugar moiety, units having one or more anionic groups. In certain embodiments, one or more linkers may be independently be absent.

In certain embodiments, the agent further comprises a PEG-moiety to alter the circulation time in blood. Such moiety can be bound to the conjugate at any suitable structural location as would be understood by one of ordinary skill in the synthesis of such compounds.

In certain embodiments, the conjugate of the PCTP-based zwitterionic metal chelator and the targeting vector has the following formula with KUE, dPSMA-617, cRGD, FAPI, octreotide, or a bombesin analog as the targeting vector.

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In some cases, one of ordinary skill in the art would understand that one or more zwitterionic groups can be replaced with another moiety which could also impart hydrophilicity to the chelator. Such hydrophilicity-imparting groups may be substituted, added, or exchanged for alternative moieties that maintain or increase aqueous solubility and hydrophilicity of the molecule (e.g., a chelator or conjugate). Non-limiting examples of suitable hydrophilic groups include poly(ethylene glycol) (PEG) chains; saccharides and sugar derivatives (e.g., glucose, galactose, mannose, sialic acid); polyols and hydroxylated moieties (e.g., glycerol, sorbitol, mannitol, xylitol, diols, triols); carboxylic acids and carboxylates; sulfonic acids and sulfonates; sulfate groups; phosphate and phosphonate groups; amide, urea, and carbamate functionalities; amine oxides; sulfoxides; betaines (e.g., carboxybetaine, sulfobetaine); quaternary ammonium groups; guanidinium-containing groups; and polar heterocycles (e.g., morpholine, piperazine, N-vinylpyrrolidone). Such moieties may be introduced in protected or salt forms and can be tethered via suitable linkers to achieve the desired hydrophilicity, charge, and solubility characteristics.

### Targeting Vectors

In some embodiments of the metal chelators of the invention, one or more zwitterionic groups of the zwitterionic metal chelator can be replaced with a targeting vector, such as cRGD, a PSMA binding vector, such as PSMA-617 or KUE, a GPI-derivative, a FAP-targeting molecule (FAP-inhibitor or FAPI), octreotide, a bombesin analog, or their corresponding homo- and hetero-dimers provided that zwitterionic metal chelator remains zwitterionic.

In certain embodiments, the targeting vector can be attached to the metal chelators of the invention through an ester linkage. In particular embodiments, the targeting vector can be attached to the metal chelators by reaction with a carboxyl-substituted phenyl group. In still other embodiments, the targeting vector can be attached to the metal chelators by the inclusion of one or more spacer groups or linker groups, wherein the spacer or linker groups may optionally include additional free carboxyl groups or zwitterionic groups for further functionality. In particular embodiments, the targeting vector is attached to the metal chelators by one or more linker groups and through an ester linkage to a carboxyl-substituted phenyl group.

In certain embodiments of the invention, the targeting ligand according to the invention can be a cyclic-RGD having the following structure:

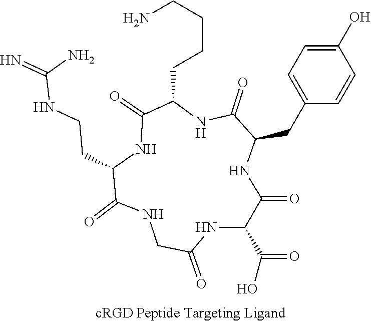 [SMILES: N=C(N)NCC[C@@H]1NC(=O)[C@H](CCCCN)NC(=O)[C@@H](Cc2ccc(O)cc2)NC(=O)[C@H](C(=O)O)NC(=O)CNC1=O]

In certain embodiments, the FAPI-targeting ligand includes NH2-FAPI-74. Specific details regarding NH2-FAPI-74 are described in: Linder et al., “Radioligands Targeting Fibroblast Activation Protein (FAP),” the entirety of which is incorporated herein by reference. In general, NH2-FAPI-74 has the following structure: the FAPI-targeting ligand includes NH2-FAPI-74

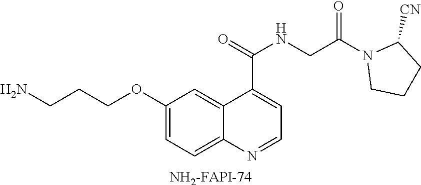

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In a particular aspect, the invention provides a zwitterionic metal chelator conjugated to a targeting vector for prostate specific membrane antigen (PSMA). For example, a targeting vector based on (((S)-5-((S)-2-((1r,4S)-4-(aminomethyl)cyclohexane-1-carboxamido)-3-(naphthalen-2-yl) propanamido)-1-carboxypentyl) carbamoyl)-L-glutamic acid, which is a derivative of PSMA-617 or Vipivotide tetraxetan. This PSMA-targeting vector is called dPSMA-617 (for “derivative of PSMA-617) in this disclosure.

An alternative PSMA-targeting vector is (((S)-5-amino-1-carboxypentyl) carbamoyl)-L-glutamic acid (KUE).

The structures of dPSMA-617 and KUE are shown in FIG. 3.

In such embodiments, a KUE or dPSMA-617-conjugated zwitterionic metal chelator includes 1~4 zwitterionic groups. In particular embodiments, the KUE or dPSMA-617-conjugated zwitterionic metal chelator includes 1-3 zwitterionic groups.

In particular embodiments, the KUE or PSMA-617-conjugated zwitterionic metal chelator has the formula:

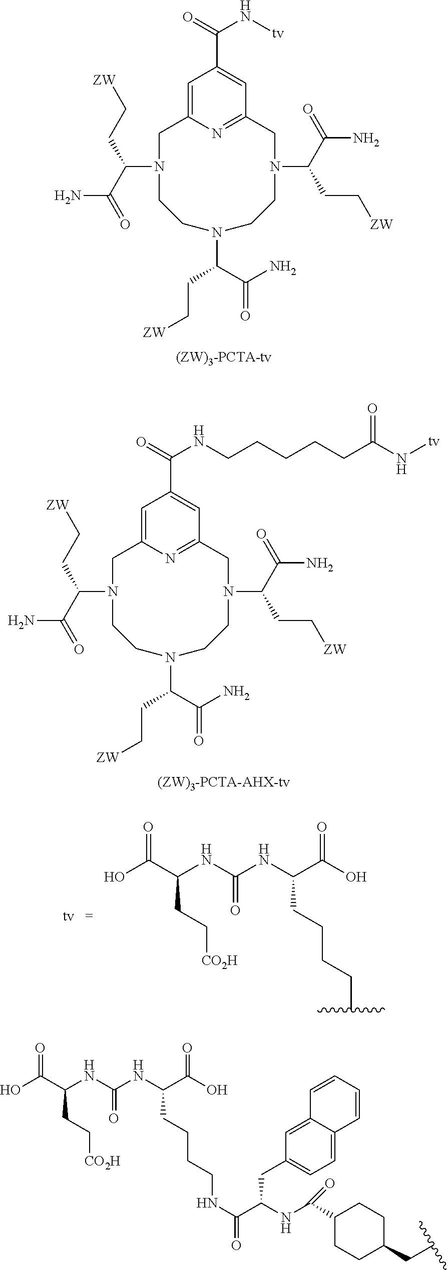

In particular embodiment, the KUE or PSMA-617-conjugated zwitterionic metal chelators can be complexed with Ga³⁺, Cu²⁺, Lu³⁺, Zr⁴⁺, Mn²⁺, Mn³⁺, Pb²⁺, Tb³⁺, Gd³⁺, or similar metal cations.

In certain embodiments, the targeting ligand includes a phosphinate mimic of N-acetylaspartylglutamic acid (GPI) or a derivative thereof. Particular derivatives of GPI can be found in U.S. Provisional Ser. No. 63/858,228 filed Aug. 5, 2025.

In certain embodiments, the GPI targeting ligand may serve as the pharmacophore component that provides selective binding to prostate-specific membrane antigen (PSMA). The GPI targeting ligand may comprise a glutamate portion linked to a phosphoramidate portion, forming a bifunctional binding unit that engages multiple sites within the PSMA active site.

In certain embodiments, the GPI targeting ligand comprises a general chemical structure, where n=0 to 3 for Regions A and D:

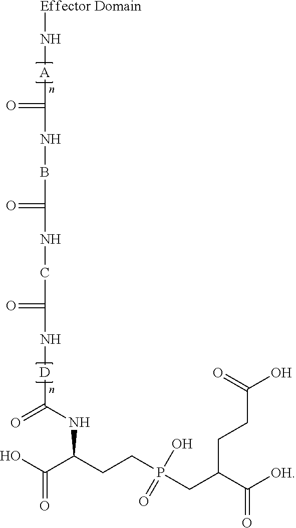

Three key examples of GPI targeting ligand includes:

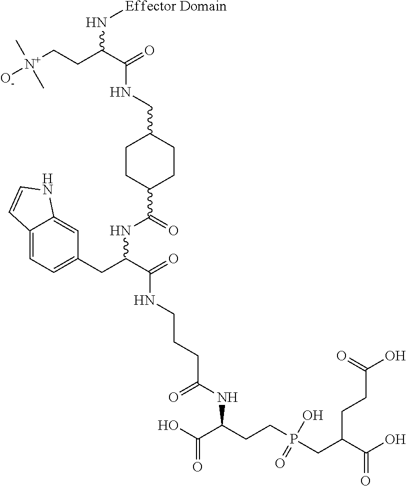

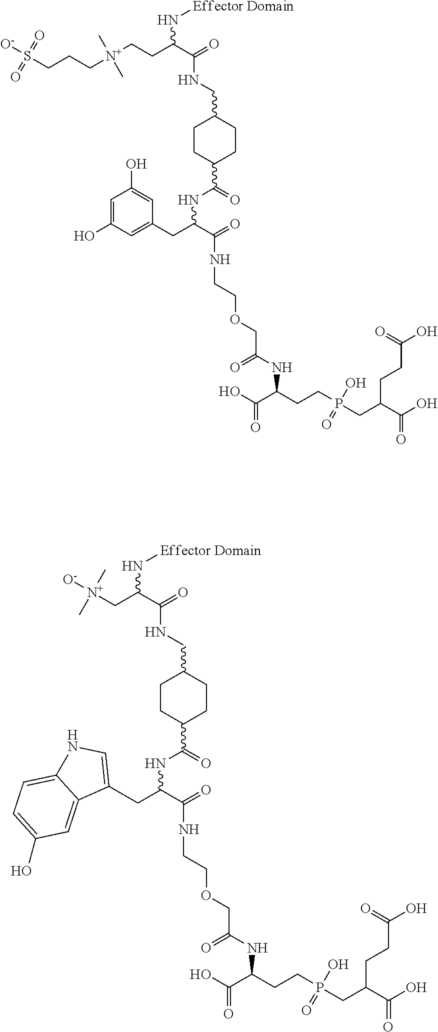

In certain embodiments, the targeting ligand includes a dimer or a multimer of one or more of KUE, dPSMA-617, GPI-derivatives, or a derivative thereof.

In general, the structure of octreotide (SEQ ID No: 5) is:

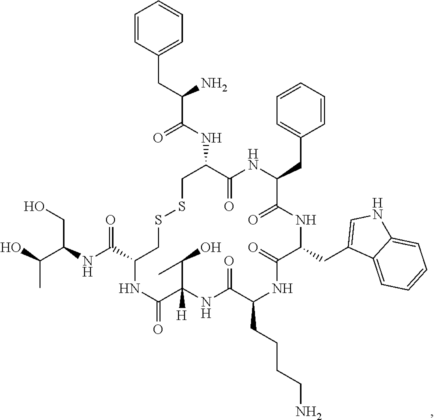 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

or a derivative thereof.

In general, the structure of a bombesin analog (SEQ ID No: 6) is:

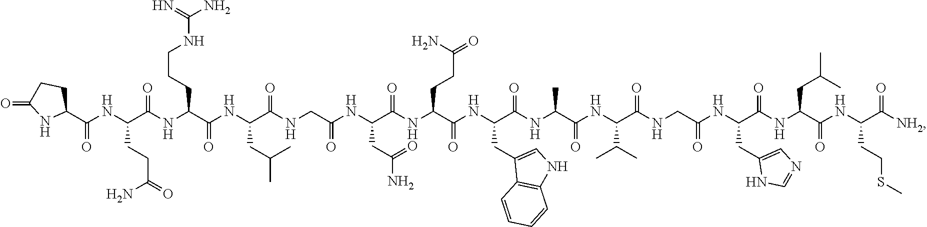 [SMILES: CSCC[C@H](NC(=O)[C@H](CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@H](CC(N)=O)NC(=O)CNC(=O)[C@H](CC(C)C)NC(=O)[C@H](CCCNC(=N)N)NC(=O)[C@H](CCC(N)=O)NC(=O)[C@@H]1CCC(=O)N1)C(C)C)C(N)=O]

or a derivative thereof.

The synthetic protocol described in Schemes 1 and 2 can be used to produce conjugated zwitterionic metal chelators based on PCTA or amide-PCTA using cRGD, a FAP-targeting molecule, octreotide, or dimers including a combination of cRGD, dPSMA-617, a FAP-targeting molecule, octreotide, and a bombesin analog. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. Depending on the reactive group of the zwitterionic metal chelator other common chemical methods may be used to conjugate the targeting vector, such as azide-alkyne-cycloadditions, nucleophilic displacements, Diels-Alder-reactions, urea and urethane formations, thiol-ene conjugations or similar conversions. For some of these conversions, derivatives of KUE, dPSMA-617, or GPI with other reactive functional groups can be used. For example, a conjugation via a copper-catalyzed azide-alkyne-cycloaddition could require either an alkyne or an azide group as the reactive group at the zwitterionic chelator and a matching alkyne or azide group as the reactive group at the targeting vector.

In certain embodiments, the targeting ligand can further include a molecular scaffold moiety to which the binding moiety and other groups can attach. In certain embodiments, the molecular scaffold moiety includes a carboxyl-substituted phenyl group which can form an ester linkage to a targeting ligand-either directly or through additional linker groups as discussed herein. In other embodiments, the molecule scaffold can bear one or more of the following: (1) a moiety designed to react with the reactive linking group of the dye to form a covalent bond, (2) a charge balancing moiety, such as any of the ionic groups described herein, and (3) a moiety that binds to the biological target. An example of a molecular scaffold is an adamantane derivative, such as described in U.S. Pat. App. Pub. No. 2006/0063834, which is incorporated herein by reference in its entirety, and illustrates the preparation of a targeting ligand that incorporates an adamantane scaffold. Specifically, the adamantane core holds (1) an amino group capable of reacting with the dye compounds, (2) a charge-balancing moiety that will neutralize a negative charge on the dye molecule, and (3) two moieties that bind to the biological target PSMA. For a description of moieties that bind to PSMA, see, Humblet, V. et al. Mol. Imaging, 2005, 4:448-62; Misra P. et al. J. Nucl. Med. 2007, 48:1379-89; Chen, Y., et al. J. Med. Chem, 2008, 51:7933-43; Chandran, S. S., et al. Cancer Biol. Ther., 2008, 7:974-82; Banerjee, S. R., J. Med. Chem. 2008, 51:4504-17; Mease, R. C., et al. Clin. Cancer Res., 2008, 14:3036-43; Foss, C. A. et al. Clin. Cancer. Res., 2005, 11:4022-8, each of which is incorporated herein by reference in its entirety. A flexible linker may function as a flexible spacer component that positions the zwitterionic chelator and any associated targeting moieties by setting the approach vector and separation distance needed for pocket engagement between the targeting moiety and the zwitterionic chelator. The flexible nature of the flexible linker may minimize steric strain while maximizing binding interactions with the target protein and maintaining optimal metal coordination geometry of the zwitterionic chelator.

The flexible linker may comprise polyethylene glycol units that provide flexibility and hydrophilicity to the overall zwitterionic chelator-based molecular construct. Polyethylene glycol units may offer biocompatibility and may reduce immunogenicity of the zwitterionic chelator compound. The number of polyethylene glycol units in the flexible linker may be varied to achieve the desired spacing between the zwitterionic chelator core and functional targeting regions of the compound while preserving the chelation properties of the zwitterionic framework.

In some cases, the flexible linker may comprise one to three polyethylene glycol units connecting the zwitterionic chelator to targeting vectors. A single polyethylene glycol unit may provide a shorter spacer length between the zwitterionic core and targeting moiety, while two or three polyethylene glycol units may provide progressively longer spacer lengths. The selection of one, two, or three polyethylene glycol units may allow for fine-tuning of the molecular geometry to optimize binding interactions with the target binding site while maintaining the zwitterionic properties and metal coordination stability of the zwitterionic chelator.

Alternatively, the flexible linker may comprise aminocarboxylic acid residues (for example, aminohexanoic acid) that serve as flexible spacer elements between the zwitterionic chelator and targeting vectors and also permit use of solid-phase synthesis. Aminocarboxylic acid residues may provide both flexibility and the ability to form hydrogen bonds with surrounding water molecules or protein residues. The aminocarboxylic acid residues may be selected based on their chain length and conformational properties to ensure compatibility with the zwitterionic chelator framework.

The flexible linker may comprise aminocarboxylic acid residues with varying carbon-chain lengths to adjust the distance between the zwitterionic chelator core and the target-binding targeting moiety. Shorter carbon-chain lengths may result in more compact molecular conformations that keep the zwitterionic chelator in close proximity to the targeting region, while longer carbon-chain lengths may allow for extended conformations. The carbon-chain length of the aminocarboxylic acid residues may be selected to position the zwitterionic chelator at the appropriate distance from the targeting moiety while preserving the chelator's coordination environment.

The length and composition of the flexible linker may be tuned to ensure that the zwitterionic chelator and any aromatic moieties are positioned within the target binding region at a distance typically on the order of 10-12 Å from the target anchor. This distance range may correspond to the spatial separation between the S1/S1′ cleft where the target anchor binds and the region where the zwitterionic chelator can be accommodated without interfering with target binding. The 10-12 Å separation may allow for simultaneous engagement of the targeting site and proper positioning of the target chelator without introducing unfavorable steric interactions that could compromise metal coordination.

The flexible alignment provided by the flexible linker may enhance binding affinity by allowing the zwitterionic chelator to adopt conformations that maximize favorable interactions with target binding sites while maintaining optimal chelator geometry. The flexibility may also maintain overall linker solubility and may prevent the formation of rigid conformations that could interfere with binding, cellular uptake processes, or the zwitterionic properties of the zwitterionic chelator system

The present disclosure leverages a modular approach that significantly enhances the efficiency and versatility of synthesizing final drug compounds. This modular strategy is particularly advantageous as it facilitates the use of standard solid-phase peptide synthesis (SPPS) techniques. By employing this approach, the invention allows for the precise and efficient assembly of peptide sequences, which are integral to the development of therapeutic agents. The compatibility with SPPS not only streamlines the synthesis process but also ensures high purity and yield of the final drug products. This innovation thus represents a significant advancement in the field of pharmaceutical manufacturing, enabling the scalable production of complex peptide-based therapeutics.

### Imaging Methods

In one aspect, the invention provides for methods of biomedical imaging of tissues or cells in a biological sample. In particular embodiment, the invention encompasses a method for detecting or imaging one or more cancer cells in a biological sample.

In some embodiments, the method is suitable for imaging of abnormal, but not malignant, tissue, such as defects of the musculoskeletal system using FAP as a targeting vector or vascular system using cRGD as a targeting vector.

In some embodiments, the method is suitable for imaging of cancer, tumor or neoplasm. In a further embodiment, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain/ens cancer (e.g., gliomas), throat cancer, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

In some embodiments, the cancer cells are adult solid tumor cells or pediatric solid tumor cells. Non-limiting examples of such cells include melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In particular embodiments, the cancer cells are prostate cancer cells.

In some embodiments, the biological sample is part or all of a subject. In some embodiments, the biological sample is obtained from a subject.

In particular embodiments, the method includes the steps of (a) contacting the biological sample with one or more of the zwitterionic metal chelators described above, wherein the zwitterionic metal chelator is coordinated to a metal atom capable of being detected by one or more conventional scanning methods.

In some embodiments, the compound is administered by parenteral, intranasal, sublingual, rectal, or transdermal delivery. In some such embodiments, the compound is administered intravenously. In some embodiments, the compound is administered intratumorally.

In some embodiments, the tumor or cell is found in a subject. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

In particular embodiments, the subject is a human.

The methods of imaging tissue or cells include the following basic steps:
(a) contacting the tissue or cells with an imaging agent comprising a zwitterionic metal chelator; and
(b) imaging the tissue or cells using positron emission tomography (PET), single-photon emission computerized tomography (SPECT), or magnetic resonance imaging (MRI).

The imaging agent described herein is a substance to that can be used to image tissues or cells, such as those of a living organism, for purposes of diagnosis, therapy, image-guided surgery, and the like. In some embodiments, the organism is a mammal, such as a human.

The imaging agents described herein generally has improved “signal-to-background ratio” (SBR) compared to presently known imaging agents. The improvement in SBR is believed to be a result of improved in vivo properties due to “charge-balancing.” SBR is a measure of the intensity of the signal obtained from a target (peak signal) over the measure of the intensity of the signal obtained nearby the target (background signal), the target being the tissues or cells targeted by the imaging agent. SBR measurements can be readily obtained through routine measurement procedures. Higher SBR values are more desirable, resulting in greater resolution of the imaged tissues. In some embodiments, the imaging agents achieve an SBR of at least about 1.1 (i.e., peak signal is at least 10% over background). In further embodiments, the imaging agents achieve an SBR of at least about 1.2, at least about 1.3, at least about 1.4, at least about 1.5, at least about 1.6, at least about 1.7, at least about 1.8, at least about 1.9, or at least about 2.0. In yet further embodiments, the imaging agents achieve an SBR of about 1.1 to about 50, about 1.5 to about 30, about 2.0 to about 20, about 2.0 to about 5.0, or about 5.0 to about 10.

The zwitterionic metal chelators of the invention generally have good solubility in substantially neutral aqueous media, and in particular, blood and blood serum. In some embodiments, the imaging agent has a solubility in 10 mM HEPES solution, pH 7.4, of at least about 10 μM. In further embodiments, the imaging agent has a solubility in 10 mM HEPES solution, pH 7.4, of at least about 15 μM at least about 20 μM, at least about 25 μM, at least about 30 μM, at least about 40 μM, or at least about 50 μM.

The zwitterionic metal chelators of the invention has significant improvements with regard to stability over time, allowing for dramatically improved operability and use for imaging and mapping. Similarly, the stability of the imaging agent allows for increased accuracy during surgery as the signal does not degrade over time. This is particularly important for complexes of Gd that are used as contrast for MRI. It has been established that Gd accumulates for long periods of time due to either instability in its metal chelator and/or inefficient elimination from the body. Zwitterionic metal chelators of Gd solve this problem by maximizing stability of the metal complex while maximizing rapid elimination from the body through renal clearance.

In another aspect, the disclosure encompasses a method of measuring and/or monitoring the effectiveness of various biological functions of a subject. In particular embodiments, the method provides for measuring the effectiveness of hepatic function, renal function, or blood pooling in a subject. In such embodiments, the zwitterionic metal chelators may be used with or without the addition of a targeting vector. The method includes the step of administering to a subject in need of measurement of a biological function with a quantifiable amount of one or more of the zwitterionic metal chelators described above; imaging the subject using positron emission tomography (PET), single-photon emission computerized tomography (SPECT), or magnetic resonance imaging (MRI); and determining the amount of the zwitterionic metal chelator present in the biological function being observed.

### Treatment, Diagnosis, and Monitoring Methods

### Cancerous Conditions

In another aspect, the disclosure encompasses a method for inhibiting the proliferation or growth of malignant or non-malignant cells. The method includes the step of contacting one or more cells with an effective amount of one or more of the zwitterionic metal chelators described above, wherein the metal atom is a radioactive metal isotope known to emit ionizing radiation in a form that would result in the death of cells that take up the analogs.

Non-limiting examples of metal isotopes that are used include Lu-177, Y-90, Ho-166, Re-186, Re-188, Cu-67, Au-199, Rh-105, Ra-223, Ac-225, As-211, Pb-212, and Th-227.

In some embodiments, the method is performed in vivo, ex vivo, or in vitro.

In some embodiments, the malignant cells are adult solid tumor cells or pediatric solid tumor cells. Non-limiting examples of such cells include melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In another aspect, the disclosure encompasses a method of diagnosing cancer in a subject. The method includes one or more of the imaging/detection steps outlined above. In the method, the biological sample is obtained from, part of, or all of a subject. If cancer cells are detected or imaged in the method steps, the subject is diagnosed with cancer. In certain embodiments, the method of diagnosing cancer in a subject is followed by a step of treating the subject diagnosed with cancer with a cancer therapy. In some embodiments, the cancer therapy is, surgery, chemotherapy or radiotherapy.

In some embodiments, the cancer that is diagnosed is an adult solid tumor or a pediatric solid tumor. Non-limiting examples of such cancer include melanoma, neuroblastoma, lung cancer, adrenal cancer, colon cancer, colorectal cancer, ovarian cancer, prostate cancer, liver cancer, subcutaneous cancer, squamous cell cancer, intestinal cancer, retinoblastoma, cervical cancer, glioma, breast cancer, pancreatic cancer, Ewings sarcoma, rhabdomyosarcoma, osteosarcoma, retinoblastoma, Wilms' tumor, and pediatric brain tumors.

In still another aspect, the disclosure encompasses a method of monitoring the efficacy of a cancer therapy in a human subject. The method includes performing one or more of the imaging/detection steps outlined above at two or more different times on the biological sample, wherein the biological sample is obtained from, part of, or all of a subject. The change in strength of the signals characteristic of the metal isotope between the two or more different times is correlated with the efficacy of the cancer therapy.

In another aspect, the disclosure encompasses a method of treatment of abnormal, but not malignant, tissue, such as defects of the musculoskeletal system using FAP as a targeting vector or vascular system using cRGD as a targeting vector. The method includes the step of contacting one or more abnormal cells with an effective amount of one or more of the zwitterionic metal chelators described above, wherein the metal atom is a radioactive metal isotope known to emit ionizing radiation in a form that would result in a therapeutic effect on the cells that take up the analogs.

In another aspect, the disclosure encompasses a method of treating a cancer by administering an effective amount of a therapeutic agent comprising a zwitterionic metal chelator complex and a pharmaceutically acceptable carrier or excipient. The method comprises a step to diagnose the cancer and a step of administering the therapeutic agent to a subject determined to be in need thereof;
wherein the step to diagnose the cancer comprises:
   contacting cells, tissues or organs of a subject with an imaging agent,
   imaging the cells, tissues, or organs of the subject using positron emission tomography (PET), single-photon emission computerized tomography (SPECT), or magnetic resonance imaging (MRI), and
   diagnosing the cancer in the cells tissues, or organs of the subject based on imaging data collected;
and wherein the metal atom complexed to the zwitterionic metal chelator is:
   a radioactive metal isotope known to emit ionizing radiation that results in the death of cells that take up the analogs;
   or a non-radioactive metal that is capable of releasing cytotoxic radiation upon irradiation with alpha emission, beta emission, neutron capture, or a combination thereof.

In still another aspect, the disclosure encompasses a method of treating a rare or childhood cancer by administering an effective amount of a therapeutic agent comprising a zwitterionic metal chelator complex and a pharmaceutically acceptable carrier or excipient. In such aspects, the therapeutic agent may further comprise one or more targeting vectors selected from the group consisting of cRGD, PSMA, FAP, octreotide, a bombesin analog, or a homomeric or heteromeric oligomers formed from their combination.

In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin. In addition, the metal atom complexed to the zwitterionic metal chelator is:
a radioactive metal isotope known to emit ionizing radiation that results in the death of cells that take up the analogs;
or a non-radioactive metal that is capable of releasing cytotoxic radiation upon irradiation with alpha emission, beta emission, neutron capture, or a combination thereof.

In particular embodiments, the rare or childhood cancer is: Acinic cell carcinoma, ACTH-secreting tumor, Actinic keratosis, Adamantinoma, Adenoid cystic carcinoma, Alveolar soft part sarcoma, Ampullary cancer, Angiosarcoma, Appendix (appendiceal) neuroendocrine (carcinoid) tumor, Askin tumor—a type of Ewing tumor (Ewing sarcoma), Bartholin gland cancer, Basaloid squamous cell carcinoma of the anus, Bowen disease, Bronchioloalveolar carcinoma, Carcinoid tumor, Carcinoma of the ampulla of Vater, Cardiac angiosarcoma, Castleman disease, Cholangiocarcinoma, Choriocarcinoma, Choroid plexus tumor, Chondrosarcoma, Chordoma, Chromophobe renal cell carcinoma, Clear cell sarcoma, Craniopharyngioma, Dermatofibrosarcoma protuberans, Desmoid tumor, Desmoplastic small round cell tumor, Dysgerminoma, Embryonal carcinoma, Endodermal sinus tumor, Endometrial stromal sarcoma, Ependymoma, Epithelial appendix (appendiceal) cancer, Epithelial-myoepithelial carcinoma, Epithelioid hemangioendothelioma (EHE), Epithelioid sarcoma, Essential thrombocythemia, Esthesioneuroblastoma (olfactory neuroblastoma), Extra-cranial malignant rhabdoid tumor (MRT), Extranodal NK/T-cell lymphoma-nasal type, Fallopian tube cancer, Fibrolamellar carcinoma, Fibromatosis, Fibromyxoid sarcoma (Evans' tumor), Fibrosarcoma Folliculotropic mycosis fungoides, Ganglioglioma, Ganglioneuroblastoma, Gastric Adenocarcinoma and Proximal Polyposis of the Stomach (GAPPS), Gastrinoma, Gastroesophageal junction (GEJ) cancer, Gestational trophoblastic disease (GTD) (hydatidiform mole; gestational trophoblastic neoplasia), Germ cell tumor, Giant cell tumor of bone, Glucagonoma, Granulomatous slack skin, Heart cancer (cardiac angiosarcoma), Hemangioendothelioma, Hemangiosarcoma, Hepatobiliary cancer, Hepatoblastoma, Hepatocellular carcinoma, Hepatoma, Hereditary diffuse gastric cancer (HDGC), Hurthle cell cancer (oxyphil cell carcinoma), Insulinoma, Islet cell tumor, Keratoacanthoma, Klatskin tumor, Large cell neuroendocrine carcinoma, Leiomyosarcoma, Leydig cell tumor, Lip cancer, Liposarcoma, Lymphomatoid papulosis, Lymphoplasmacytic lymphoma, Malignant mesenchymoma, Malignant mixed mullerian tumor, Malignant peripheral nerve sheath tumor (MPNST), Malignant rhabdoid tumor of the kidney, Medulloepithelioma, Meningioma, Mesoblastic nephroma, Metaplastic cancer of the breast, Monoclonal gammopathy of undetermined significance (MGUS), Mouth cancer, Mucinous cystic neoplasm, Mucoepidermoid carcinoma, Muscle cancer (myosarcoma), Myoepithelial carcinoma, Mycosis fungoides, Myelofibrosis, Myxofibrosarcoma, Nephroblastoma, Neuroendocrine carcinoma of the skin, NUT carcinoma, Oat cell cancer, Occult primary cancer, Ocular or intraocular melanoma, Olfactory neuroblastoma (esthesioneuroblastoma), Oligodendroglioma, Oncocytic carcinoma, Ovarian small cell cancer, Paget disease, Pagetoid reticulosis, Paraganglioma, Parathyroid cancer, Periosteal osteosarcoma, Peripheral primitive neuroectodermal tumor (PPNET), Pheochromocytoma, Phyllodes tumor, Pineoblastoma, Plasmacytoma, Polycythemia vera, Polymorphous low-grade adenocarcinoma, Primary cutaneous lymphoma, Primary peritoneal carcinoma, Prolactinoma (lactotroph adenoma), Renal cell carcinoma, Sarcomatoid carcinoma (carcinosarcoma), Schwannoma, Sclerosing epithelioid fibrosarcoma, Sebaceous carcinoma, Seminoma, Sertoli cell tumor, Sezary syndrome, Sinus cancer, Skin adnexal tumors, Solid pseudopapillary neoplasm, Solitary fibrous tumor, Solitary plasmacytoma, Somatostatinoma, Spermatocytic seminoma, Spindle cell neoplasm, spindle cell tumor, spindle cell carcinoma, spindle cell sarcoma, Subcutaneous panniculitis-like T-cell lymphoma, Synovial sarcoma, T-cell lymphoma, Teratoma, Throat cancer, Thymoma, Tongue cancer, Tonsil cancer, Trabecular cancer, Translocation renal cell carcinoma, Transitional cell carcinoma (urothelial carcinoma), Undifferentiated pleomorphic sarcoma, Urachal cancer, Urethral cancer, Urothelial carcinoma (transitional cell carcinoma), Uterine cancer, Verrucous carcinoma, VIPoma, Vocal cord/voice box cancer, Womb cancer, or Yolk sac tumor.

### Non-Cancerous Conditions

In another aspect, the invention provides a method of treating a non-cancerous condition in a subject in need thereof, the method comprising: administering to the subject an effective amount of a therapeutic agent comprising a zwitterionic metal chelator complex and a pharmaceutically acceptable carrier or excipient. In certain embodiments, the metal atom complexed to the zwitterionic metal chelator is:
a radioactive metal isotope known to emit ionizing radiation that results in a therapeutic effect on the subject.
or a non-radioactive metal that is capable of releasing therapeutic radiation upon irradiation with alpha emission, beta emission, neutron capture, or a combination thereof.

In certain embodiments, the non-cancerous condition is a musculoskeletal disorders or a tissue hypertrophy disorder. In still other embodiments, the non-cancerous condition is myocardial infarction, atherosclerosis, or fibro-inflammatory disease (including, but not limited to interstitial lung disease, rheumatoid arthritis, liver fibrosis, and keloids).

In still other embodiments, the subject is a human.

### Efficacy Measurements of Biological Systems.

In another aspect, the disclosure encompasses a method of measuring the efficacy of a biological system of a subject. In particular embodiments, the biological system to be measured or monitored is the renal system, the hepatic system, or the blood pool.

In particular embodiments, the method of measuring the efficacy of a biological system includes:
(a) administering a quantifiable amount of a diagnostic agent comprising a zwitterionic metal chelator complex and a pharmaceutically acceptable carrier or excipient to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In a particular aspect, the disclosure encompasses a method of measuring the efficacy of renal function a subject. In particular embodiments, the method comprises:
(a) administering a quantifiable amount of a diagnostic agent comprising a zwitterionic metal chelator complex and a pharmaceutically acceptable carrier or excipient to the subject;
(b) imaging the subject with positron emission tomography (PET), single-photon emission computerized tomography (SPECT), or magnetic resonance imaging (MRI); and
(c) determining the amount of therapeutic agent present in the biological system being observed in the subject.

In another aspect, the invention provides a method of quantifying the glomerual filtration rate of a subject. In particular embodiments, the method comprises:
(a) administering a quantifiable amount of a diagnostic agent comprising a zwitterionic metal chelator complex and a pharmaceutically acceptable carrier or excipient to the subject; and
(b) determining the amount of diagnostic agent present in the blood and urine of the subject as a function of time using either measurements of each bodily fluid or imaging the subject with positron emission tomography (PET), single-photon emission computerized tomography (SPECT), or magnetic resonance imaging (MRI).

### Radiosurgery

Radiosurgery is a known method of treating targets in the body. During radiosurgery, the target is bombarded with a series of X-ray beams fired from various different positions and orientations by using a radiation delivery system, to affect the tumor biology using the cumulative radiation dose at the target. The radiation can be delivered invasively in conjunction with traditional scalpel surgery, or through a percutaneous catheter. CyberKnife™ (Accuray Inc.) and Trilogy™ (Varian Medical Systems) are two such radiation delivery systems. Advances in stereotactic surgery have provided increased accuracy in registering the position of tissue targeted for treatment and a radiation source. For example, see U.S. Pat. Nos. 6,351,662 and 6,402,762. Stereotactic radiosurgery systems may be commercially available from ACCURAY, INC. of Sunnyvale, Calif., and BRAINLAB. The Accuray Cyberknife™ stereotactic radiosurgery system has reportedly been used to provide targeted, painless, and fast treatment of tumors.

In one aspect, the invention provides a radiosurgical method for treating a patient body, the method comprising:
receiving a desired lesion pattern and planned radiation distribution;
administering an effective amount of a diagnostic agent comprising a zwitterionic metal chelator complex and a pharmaceutically acceptable carrier or excipient to the subject to effectively image the desired lesion pattern; and
performing surgery on the desired lesion pattern to treat the patient body.

In certain embodiments, the surgery can be using scalpel surgery or a stereotactic radiosurgery system to administer a cumulative radiation dose.

In other embodiments, the zwitterionic metal chelator of the diagnostic agent further comprises one or more targeting vectors wherein the one or more targeting vectors are cRGD, KUE, dPSMA-617, a GPI-derivative, a FAP-targeting molecule, octreotide, a bombesin analog, or homomeric or heteromeric oligomers formed from their combination. In certain embodiments, the targeting ligand includes one or more of LyP-1 peptide having a sequence of CGQKRTRGC (SEQ ID NO: 1) and binding to P32 for diagnosing/treating melanoma; K237 peptide having a sequence of HTMYYHHYQHHL (SEQ ID NO: 2) and binding to VEGFR-2 for diagnosing/treating breast tumor; IL4RPep-1 peptide having a sequence of CRKRLDRNC (SEQ ID NO: 3) and binding to IL4R for diagnosing/treating lung tumor, breast tumor, colon tumor; mUNO peptide having a sequence of CSPGAK (SEQ ID NO: 4) and binding to CD206 for diagnosing/treating breast tumor; folate receptors for diagnosing/treating ovarian and lung cancer; GE11, a dodecapeptide, binding to epidermal growth factor receptor (EGFR or ErbB1) for diagnosing/treating tumors of epithelial origin.

In still other embodiments, the desired lesion pattern is received from a user interface of a treatment planning module. Such planning modules can be pre-programmed with specifications for various disease states and cancerous conditions. Alternatively, specifications for desired lesion patterns can be identified and produced using artificial intelligence data.

### Toxic Metals

The zwitterionic metal chelators described herein can also be used as a treatment for removal of toxic or excess metals in a subject. In such methods, the zwitterionic metal chelators are administered without a metal coordinated thereto. The chelators act to bind excess or toxic metals in the subject which are then cleared along with the chelators through the kidneys and the renal system. The use of zwitterionic metal chelators for this method allows for faster removal of excess metals from a subject with increased clearance times and less damage to the liver and other organs.

As such, in one aspect, this disclosure encompasses a method for removing toxic or excess metals in a subject in need of such treatment, the method comprising:
(a) administering a therapeutically effective amount of a therapeutic agent to the subject,
wherein the therapeutic agent comprises a metal chelator having one or more zwitterionic groups which is not coordinated to a metal or metal isotope, and a pharmaceutically acceptable carrier or excipient.

In still other embodiments, the toxic or excess metals are lead, mercury, arsenic, cadmium, thallium, iron, zinc, chromium, manganese, aluminum, cobalt, selenium, beryllium, lithium, silver, or tin.

In still other embodiments, the toxic or excess metals are lead, mercury, arsenic, cadmium, thallium, iron, zinc, chromium, manganese, aluminum, cobalt, selenium, beryllium, lithium, silver, or tin.

### Other Applications—Site-Directed Labeling

The zwitterionic metal chelators of the invention can be conjugated to various biomolecules and targeting agents through site-directed labeling techniques. These conjugation methods allow for the precise attachment of the zwitterionic metal chelators to specific sites on target molecules while preserving the biological activity and binding properties of both the chelator and the target molecule.

The conjugation of zwitterionic metal chelators to monoclonal antibodies represents a particularly useful application of site-directed labeling. Monoclonal antibodies provide highly specific targeting capabilities for various disease states and cellular targets. In some embodiments, the zwitterionic metal chelators may be conjugated to monoclonal antibodies through lysine residues using NHS ester chemistry. The NHS ester group of the chelator reacts with primary amine groups on lysine residues to form stable amide bonds. In other embodiments, conjugation may occur through cysteine residues using maleimide chemistry, where the maleimide group reacts with sulfhydryl groups to form thioether linkages.

Site-specific conjugation techniques may be employed to achieve more controlled and homogeneous conjugates. In certain embodiments, engineered cysteine residues may be introduced at specific locations on the antibody to provide defined conjugation sites. Alternatively, unnatural amino acids containing reactive functional groups may be incorporated into the antibody during expression to enable site-specific attachment of the zwitterionic metal chelators.

The zwitterionic metal chelators may also be conjugated to other protein-based targeting agents including antibody fragments such as Fab, F(ab′) 2, scFv, diabodies, and nanobodies. These smaller protein constructs may offer advantages in terms of tissue penetration and clearance properties while maintaining specific binding capabilities.

The conjugation chemistry may also accommodate peptides, proteins, nucleic acids, carbohydrates, and synthetic polymers. For peptide conjugation, standard peptide coupling reagents such as carbodiimides (EDC, DCC), coupling agents (HATU, HBTU), and activating agents may be employed. Nucleic acid conjugation may utilize phosphoramidite chemistry or click chemistry approaches such as copper-catalyzed azide-alkyne cycloaddition (CuAAC) or strain-promoted azide-alkyne cycloaddition (SPAAC).

The site-directed labeling approach allows for the preparation of conjugates with improved pharmacokinetic properties, enhanced target specificity, and reduced off-target effects compared to non-specifically labeled constructs. The zwitterionic nature of the metal chelators may contribute to reduced non-specific binding and improved clearance properties of the resulting conjugates

### Other Applications—Agricultural and Chemical Processes

Zwitterionic metal chelators improve solubility and minimize non-specific interactions. It is believed that these improved properties result from the balancing of formal charges on the metal chelator, rendering a polyionic yet “charge-balanced” molecule having a net charge that is neutral or close to neutral, with an extended sphere of hydration and better isolation of the metal.

The zwitterionic metal chelators described herein can also be used in agricultural systems. In particular embodiments, zwitterionic metal chelators described herein can be used to supply micronutrients (trace metals such as iron, zinc, manganese, and copper) to various crops and plants. These micronutrients can help prevent drought and other diseases, particularly those caused by over-usage of certain fertilizers (particularly phosphorous fertilizer). Similarly, the zwitterionic metal chelators described herein can be used to supply various herbicidal formulations which are otherwise insoluble.

Similarly, the zwitterionic metal chelators described herein can also be used in chemical processes. As with the exposure to toxic metals described herein, certain chemical processes require the sequestration and removal of excess metals, such as metal catalysts. As such, the zwitterionic metal chelators described herein can also be used in such chemical process to remove insoluble metal impurities and/or unwanted metal components from the chemical process.

In particular, the zwitterionic metal chelators described herein have an advantage as compared to traditional metal chelators in the polarity of the zwitterionic molecules. As a result of this polarity, the zwitterionic metal chelators described herein can be used in aqueous solution, e.g. to increase solubility of a metal, and can then be precipitated from solution by addition of less polar solvents. This precipitation allows for the capture and subsequent disposal of undesired metals, —for example in capturing iron from wastewater (urban mining). In other embodiments, the zwitterionic metal chelators described herein which include sulfobetaines, precipitation can be induced by salt addition.

### Dosage Forms and Administration Methods

In the methods of the invention, the zwitterionic metal chelators are administered at a predetermined dosage. The predetermined dosage amount is not particularly limited provided that the predetermined dosage is administered at least a minimum amount capable of being cleared by the kidneys within twelve hours. In particular embodiments utilizing non-radioactive techniques, the predetermined dosage is 0.5 mg/kg of body weight; 0.25 mg/kg of body weight; 0.1 mg/kg of body weight; 0.05 mg/kg of body weight; 0.01 mg/kg of body weight; 0.005 mg/kg of body weight; or 0.001 mg/kg of body weight. In other embodiments, the predetermined dosage is 5.0 mg; 2.5 mg, 1.0 mg; 0.75 mg; 0.5 mg; 0.25 mg; or 0.1 mg. In particular embodiments utilizing radioactive techniques, the dose will be limited by dose-limiting toxicity. In general, effective doses will be in the range of 0.1 mCi to 20 mCi for imaging, and 0.1-10 mCi/kg for therapy.

In some embodiments, a detectably effective amount of the zwitterionic metal chelators of the presently disclosed methods is administered to a subject. In accordance with the presently disclosed subject matter, “a detectably effective amount” of the imaging agent is defined as an amount sufficient to yield an acceptable image using equipment which is available for clinical use. A detectably effective amount of the agent may be administered in more than one injection. The detectably effective amount of the imaging agent can vary according to factors such as the degree of susceptibility of the individual, the age, sex, and weight of the individual, idiosyncratic responses of the individual, the dosimetry, and instrument and film-related factors. Optimization of such factors is well within the level of skill in the art.

The administration of the agent of the invention can be by any means described herein and as generally acceptable to the patient and one of ordinary skill in the art. In particular, the administration of the charge-balanced imaging agent is intravenous.

The predetermined target amount is dependent on a variety of factors including, but not limited to the type of tumor or condition to be observed and the location of any cells desired to be imaged. As such, the predetermined amount is set by one of ordinary skill in the art prior to administration of the agent. Such factors include, but are not limited to, the determined dosage amount, the height, weight, age, body mass index, and gender of the patient or any combination thereof.

In particular embodiments utilizing non-radioactive techniques, when the predetermined dosage amount is between about 2.5 mg and about 5.0 mg or greater, the predetermined target amount is 50% of the pre-determined dosage amount. In other embodiments, when the predetermined dosage amount is between about 0.5 mg and about 2.5 mg, the predetermined target amount is 60% of the pre-determined dosage amount. In still other embodiments, when the predetermined dosage amount is about 0.5 mg or less, the predetermined target amount is 80% of the pre-determined dosage amount.

Any route of administration may be suitable for administering the disclosed agents to a subject. In one embodiment, the disclosed zwitterionic metal chelators may be administered to the subject via intravenous injection. In another embodiment, the disclosed zwitterionic metal chelators may be administered to the subject via any other suitable systemic deliveries, such as parenteral, intranasal, sublingual, rectal, or transdermal administrations.

In another embodiment, the disclosed zwitterionic metal chelators may be administered to the subject via intraperitoneal injection or IP injection.

In another embodiment, the disclosed zwitterionic metal chelators may be administered to the subject via intratumoral injection.

For treatment of non-malignant or malignant tissues using neutron capture therapy, the subject is bombarded with neutrons at the appropriate energy after administration of the (typically targeted) zwitterionic chelator and adequate time for biodistribution, binding, and clearance of unbound dose.

The disclosure also includes methods of using pharmaceutical compositions comprising one or more of the disclosed zwitterionic metal chelators in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for parenteral, intranasal, sublingual or rectal administration, or for administration by inhalation or insufflation.

For preparing solid compositions such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a zwitterionic metal chelator of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid pre-formulation composition is then subdivided into unit dosage forms of the type described above containing from 0.1 to about 500 mg of the active ingredient of the present invention. Typical unit dosage forms contain from 1 to 100 mg, for example, 1, 2, 5, 10, 25, 50 or 100 mg, of the active ingredient. The tablets or pills of the novel composition can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the zwitterionic metal chelators may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium caboxymethylcellulose, methylcellulose, polyvinylpyrrolidone or gelatin.

The disclosed zwitterionic metal chelators are particularly useful when formulated in the form of a pharmaceutical injectable dosage, including in combination with an injectable carrier system. As used herein, injectable and infusion dosage forms (i.e., parenteral dosage forms) include, but are not limited to, liposomal injectables or a lipid bilayer vesicle having phospholipids that encapsulate an active drug substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection.

Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

The present invention also provides packaged pharmaceutical compositions comprising a pharmaceutical acceptable carrier and a zwitterionic metal chelator of the invention. In certain embodiments the packaged pharmaceutical composition will comprise the reaction precursors necessary generate the zwitterionic metal chelator of the invention upon combination with a metal or radiolabeled precursor.

The invention also permits the zwitterionic metal chelator to be provided in a non-radioactive “kit” form comprised of the non-radioactive zwitterionic chelator/targeting vector molecule, a preparation apparatus (for example, a hot plate), and quality control devices (for example, a test strip or equivalent). The user would add the radioactive metal to create the final injected drug immediately before use as an imaging agent and/or therapeutic agent. In this way, the kit can be manufactured and shipped in a non-radioactive form, and the radioactive metal is added at the site. The non-radioactive zwitterionic chelator/targeting vector molecule may be provided in solution or in lyophilized form. When the imaging agent and carrier of the kit are in lyophilized form, the kit may optionally contain a sterile and physiologically acceptable reconstitution medium such as water, saline, buffered saline, and the like.

The kit may provide a zwitterionic metal chelator of the invention in solution or in lyophilized form, and these components of the kit of the invention may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form.

Determination and optimization of such stabilizers and stabilization methods are well within the level of skill in the art. When the targeting molecule/chelating agent of this embodiment are in lyophilized form, the kit may optionally contain a sterile and physiologically acceptable reconstitution medium such as water, saline, buffered saline, and the like. The amounts of unlabeled targeting molecule/chelating agent, auxiliary molecule, and reducing agent in this embodiment are optimized in accordance with the methods for making the cardiovascular imaging agent set forth above. Radionuclides may be combined with the unlabeled targeting molecule/chelating agent and the reducing agent for a time and at a temperature sufficient to chelate the radionuclide to the targeting molecule/chelating agent, and the imaging agent thus formed is injected into the patient.

Imaging agents of the invention may be used in accordance with the methods of the invention by one of skill in the art. Images can be generated by virtue of differences in the spatial distribution of the imaging agents which accumulate at a site. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions. A particularly useful imaging approach employs more than one imaging agent to perform simultaneous studies.

Generally, the diagnostic compositions are administered in doses effective to achieve the desired signal strength to enable detection. Such doses can vary, depending upon the organs or tissues to be imaged, and the imaging equipment being used. For example, Zeheer et al., Nature Biotechnology, 19, 1148-1154 (2001) uses 0.1 μmol/kg as a dose for IRDye78 conjugates in vivo. The diagnostic compositions can be administered to a patient systemically or locally to the organ or tissue to be imaged, and then the patient is subjected to the imaging procedure.

Preferably, a detectably effective amount of the imaging agent of the invention is administered to a subject. In accordance with the invention, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, the dosimetry. Detectably effective amounts of the imaging agent of the invention can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

### EXAMPLES

### Example 1: Preparation of Zwitterionic Metal Chelators

### Bn₃PCTAZA

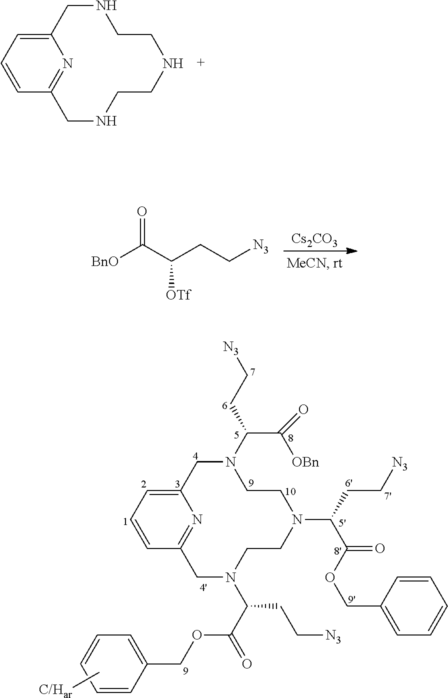

To a suspension of pyclene (128 mg, 620 μmol, 1.00 eq.) in dry MeCN (5 mL) was added Cs₂CO₃ (707 mg, 2.17 mmol, 3.50 eq.) followed by the dropwise addition of a solution of the triflate (752 mg, 2.05 mmol, 3.30 eq.) in MeCN (15 mL). The reaction mixture was stirred at room temperature for 46 h. Afterwards the solvent was removed in vacuo and the residue was taken up in a mixture of sat. aq. NaHCO₃ solution (25 mL) and CH₂Cl₂ (20 mL). The phases were separated and the aq. phase was extracted with CH₂Cl₂ (5×20 mL). The combined org. phases were dried over Na₂SO₄ and the solvent was removed in vacuo. The crude product was obtained as an orange oil. The crude product was purified via flash chromatography (loaded as solution in CH₂Cl₂/MeOH/Et₃N 95:4.5:0.5 (1.2 mL), silica gel, MN Cartridge BT15, CH₂Cl₂/MeOH/Et₃N 95:4.5:0.5 isocratic gradient). The product was obtained as an orange oil. Due to the oily consistency of the product solvents were hardly removed via rotavap completely. Therefore the product was taken up in MeCN/H₂O 80:20 and lyophilized. After lyophilisation the product was obtained as an orange oil (408 mg, 77%).

R_{f} (CH₂Cl₂/MeOH/Et₃N 95:4.5:0.5)=0.6.

t_{R}=16.5 min (Gradient and column type see below in Tab. 1).

¹H (C₆D₆, 600 MHz): δ (ppm)=7.25 (m, 2H, H-2), 7.16-7.06 (m, 15H, Hₐᵣ), 6.73 (m, 1H, H-1), 5.01 (m, 4H, H-9), 4.90 (m, 2H, H-9′), 3.88 (d, ³J_{H,H}=11.86 Hz, 2H, H-4), 3.73 (d, ³J_{H,H}=11.65 Hz, 2H, H-4), 3.40 (m, 2H, H-5), 3.10 (m, 1H, H-5′), 3.08 (m, 2H, H-7, H-7′), 3.02 (m, 4H, H-10), 2.98-2.77 (m, 4H, H-7, H-7′), 2.30 (m, 4H, H-9), 1.88-1.45 (m, 6H, H-6, H-6′).

¹³C (C₆D₆, 600 MHz): δ (ppm)=172.6 (C-8′), 172.1 (C-8), 158.6 (C-3), 137.3 (C-1), 136.5 (Cₐᵣ), 128.9 (Cₐᵣ), 128.8 (Cₐᵣ), 128.7 (Cₐᵣ), 128.6 (Cₐᵣ), 128.4 (Cₐᵣ), 122.7 (C-2), 66.4 (C-9), 66.2 (C-9′), 64.0 (C-5), 63.3 (C-5), 57.9 (C-4, C-4′), 53.3 (C-9), 51.1 (C-10), 48.4 (C-7), 48.3 (C-7′), 30.3 (C-6′), 29.9 (C-6).

HR-MS (ESI, positive) m/z=858.4160 (calc, for C₄₄H₅₁N₁₃O₆: 858.4158 [M+H]⁺).

TABLE 1
Gradient for Macherey Nagel NUCLEODUR C18
Gravity-SB, 3 μm, 150 × 2 mm.

| | MeCN + 0.1% | H₂O + 0.1% |
|---|---|---|
| Time | Formic acid | Formic acid |
| 0 min | 98% | 2% |
| 3 min | 98% | 2% |
| 18 min | 2% | 98% |
| 22 min | 2% | 98% |
| 23 min | 98% | 2% |
| 29 min | 98% | 2% |

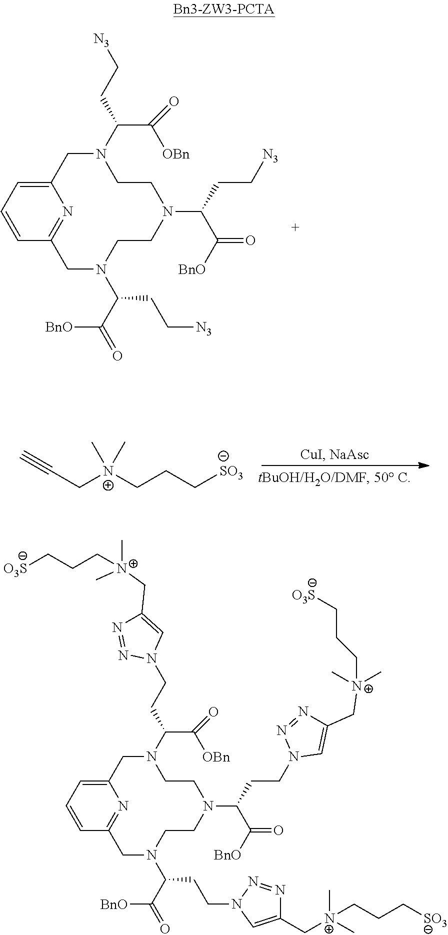

A solvent mixture of t-BuOH/H₂O/DMF 2:2:1 (5 mL) was degassed via bubbling N₂ through the solution with a cannula for 15 minutes. Afterwards CuI (8.90 mg, 47.0 μmol, 0.10 eq.) and NaAsc (46.2 mg, 233 μmol, 0.50 eq.) were added and the degassing process was allowed to proceed for further 5 minutes while stirring. Following Bn₃PCTAZA (400 mg, 466 μmol, 1.00 eq.) as a solution in DMF (2 mL) and ZW-C₃-Alkine (325 mg, 1.59 mmol, 3.40 eq.) were added and the reaction mixture was warmed to 50° C. The reaction mixture was stirred at this temperature for 24 h. Solvents were removed in vacuo and the crude product was obtained as an orange oil. The crude product was purified via flash chromatography (RP C18ec silica, MN cartridge RS15ec, H₂O/MeCN 98:2 to 2:98 in 8 cv). After lyophilisation the product was obtained as colourless lyophilisate.

### Example 2: Imaging of Organisms

For in vivo characterization, 40 μmol/g (average 10 nmol) of a zwitterionic metal chelator complex can be injected IV into 25 g athymic nude mice harboring xenograft human tumors. Simultaneous color video and positron emission tomography (PET), single-photon emission computerized tomography (SPECT), or magnetic resonance tomography (MRT) can be acquired pre-injection, every 1 sec for the first 20 sec then every 1 min for 2 h. Camera acquisition can be held constant (typically 100 msec) and chosen to ensure that all intensity measurements are within a linear range. Blood can be sampled at 0, 1, 2, 5, 10, 15, 30, 60, and 120 min via tail vein. Intensity-time curves for all major organs and tissues can be quantified. The peak intensity and time can be determined for each tumor/tissue/organ, along with the intensity in each at 1 h post-injection.

### Example 3: Gd—SB₃-PCTA and Gd-NOx₃-PCTA

### Synthesis of Zwitterionic Chelators and Corresponding Complexes

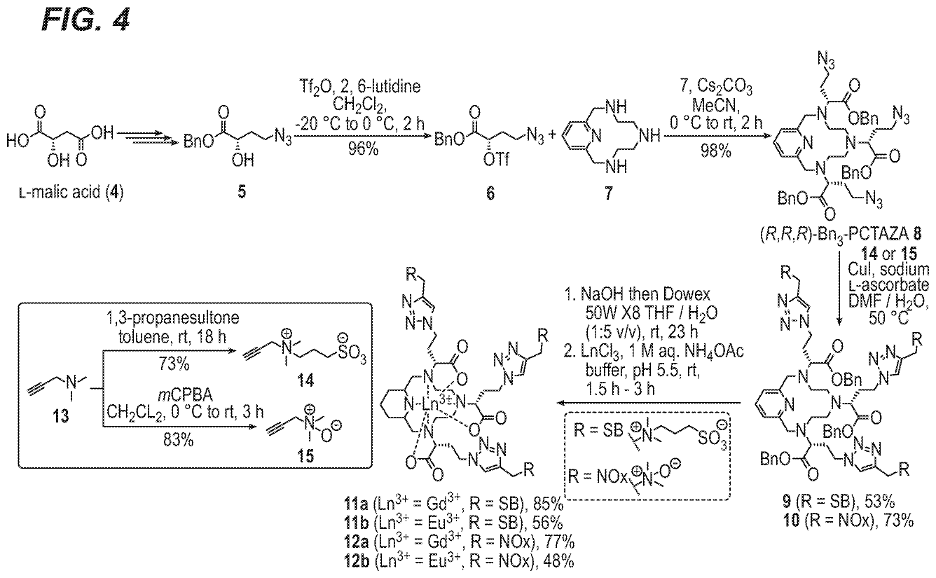

A new clickable chelator (R,R,R)-Bn₃-PCTAZA 8 (Scheme 3-FIG. 4) was prepared via alkylation of 3,6,9-triaza-1 (2,6)-3,6,9,15-tetraazabicyclo[9.3.1]pentadeca-1 (15), 11,13-triene (7, pyclen) with triflate(S)-6 using a modified procedure for the alkylation of cyclen. (E. Kriemen, E. Ruf, U. Behrens and W. Maison, Chem. Asian J., 2014, 9, 2197-2204.) Triflate(S)-6 was available in enantiomerically pure form from alcohol(S)-5, which was prepared from L-malic acid in five steps. (E. Kriemen, E. Ruf, U. Behrens and W. Maison, Chem. Asian J., 2014, 9, 2197-2204; and S. E. Denmark and S. M. Yang, J. Am. Chem. Soc., 2004, 126, 12432-12440). Alkylation of pyclen 7 with triflate(S)-6 gave the azide-functionalised chelator (R,R,R)-Bn₃-PCTAZA 8 in high yield. The yield of the alkylation step increased substantially when the triflate was added at 0° C. instead of room temperature. (R,R,R)-Bn₃-PCTAZA 8 was prepared in gram quantities with this procedure. The alkylation of pyclen with triflate 8 gives stereoisomerically pure (R,R,R)-Bn₃-PCTAZA 8 according to NMR and HPLC. The modification of (R,R,R)-Bn₃-PCTAZA 8 via CuAAC (click-chemistry) was performed according to an established protocol for the preparation of zwitterionic DOTA-derivatives with the alkynes 14 and 15. In the case of the sulfobetaine alkyne 14, 3.30 equiv. were sufficient to achieve complete conversion to Bn₃—SB₃-PCTA 9. The moderate isolated yield of 53% is a consequence of the high polarity of the compound, which led to problems in the chromatographic separation of Bn₃-SB₃-PCTA 9 from salts. HPLC-MS analysis of the crude product indicated quantitative formation of 9 with no significant formation of side products. In contrast, a large excess (18.0 eq) of alkyne N-oxide 15, was required to achieve complete and clean conversion to Bn₃-NOx₃-PCTA 10.

The benzyl esters of Bn₃-SB₃-PCTA 9 and Bn₃-NOx₃-PCTA 10 were hydrolysed under alkaline conditions using NaOH in a THF/H₂O mixture. The reaction mixture was neutralised by treatment with an acidic ion-exchange resin, which was subsequently removed via filtration, affording the zwitterionic chelators SB₃-PCTA and NOx₃-PCTA in almost quantitative yields. Complexation was performed with GdCl₃ or EuCl₃ at room temperature in aqueous 1 M NH₄OAc buffer (pH 5.5). The resulting complexes Gd—SB₃-PCTA 11a and Gd-NOx₃-PCTA 12a were obtained in good yields over two steps. The complexation was monitored by HPLC-MS, and notably, no free ligand was detected after 10 minutes, indicating a rapid and efficient complexation of both cations.

Complexes Eu—SB₃-PCTA 11b and Eu-NOx₃-PCTA 12b are important model compounds for physicochemical characterization. In particular, Eu(III) complexes allow determination of the inner-sphere hydration number q via luminescence lifetime measurements. A. Beeby, I. M. Clarkson, R. S. Dickins, S. Faulkner, D. Parker, L. Royle, A. S. de Sousa, J. A. G. Williams and M. Woods, J. Chem. Soc., Perkin Trans. 2, 1999, DOI: D

### General Considerations

The sulfobetaine alkyne 14 was synthesized according to the procedure reported by Niu et al., while the synthesis of the alkyne N-oxide 15 has also been previously described in the literature. (S.-L. Niu, G. Ulrich, P. Retailleau, J. Harrowfield and R. Ziessel, Tetrahedron Lett., 2009, 50, 3840-3844.) The alkyne N-oxide 15 was obtained in a stable and analytically pure form by the oxidation of N,N-dimethylpropargylamine (13) with meta-chloroperoxybenzoic acid (mCPBA) following a modified version of the procedure reported by Galán et al. (A. Galan, G. Gil-Ramirez and P. Ballester, Org. Lett., 2013, 15, 4976-4979.). As the alkyne N-oxide 15 has been reported to be unstable even at low temperatures but was found to be stable in aqueous solution, we utilized immediate dissolution in water upon isolation, yielding a 3 M aqueous stock solution. This solution proved to be stable at 4° C. over at least two months and provided a convenient concentration for CuAAC reactions in mixed DMF/H₂O systems, in which all reactants were sufficiently soluble. All commercially available reagents and starting materials were purchased from Sigma Aldrich, TCI, abcr or BLDpharm and were used without further purification. Gadopiclenol 3 was purchased as Vueway® from Bracco Imaging SpA as a 0.5 mmol/mL aqueous solution. Non-deuterated solvents in HPLC grade were purchased from VWR chemicals and deuterated solvents were purchased from Deutero GmbH. Water was purified using an ELGA PURELAB Classic UV water system. Reactions were monitored via HPLC-MS or TLC (Macherey Nagel TLC aluminum sheets, ALUGRAMSIL G UV254, 2.5 cm×7.5 cm). Spots were visualized under UV light and/or by staining with a basic aqueous KMnO₄ solution.

Medium pressure liquid chromatography was performed on automated systems using prepacked cartridges (Interchim). For the use of alkaline alumina an empty cartridge was filled with alkaline alumina for chromatography. Normal phase chromatography was performed using a Biotage Isolera Prime system and for reversed flash chromatography an Interchim PuriFlash 430 system with MeCN/H₂O containing 0.1% formic acid as the mobile phase.

NMR analyses were performed on Bruker Avance III HD 600 MHz and Bruker Avance I 400 MHz spectrometers. Chemical shifts (8) are expressed in parts per millions (ppm). High-resolution mass spectrometry (HRMS) was performed on an Agilent 6230 ESI-TOF coupled with an Agilent HPLC 1200 series HPLC system. Analytical HPLC-MS was performed on an Agilent HPLC system 1260 Infinity II with a Macherey Nagel NUCLEODUR C18 Gravity-SB, 3 μm, 100×2 mm column linked to a Bruker Ion Trap mass spectrometer with an ESI ionization source.

### General Synthesis Methods

Triflate 6. To a solution of(S)-benzyl-4-azido-2-hydroxybutanoat (5) (1.54 g, 6.57 mmol, 1.00 eq.) in CH₂Cl₂ (65 mL) at −20° C. was added 2,6-lutidine (1.14 mL, 9.85 mmol, 1.50 eq.) followed by the dropwise addition of trifluoromethanesulfonic anhydride (1.55 mL, 9.20 mmol, 1.40 eq.). During the addition the solution became orange, then redish. The solution was stirred at −20° C. for 1 h, the cooling bath was switched to an ice bath and the solution was stirred at 0° C. for 1 h. All volatiles were removed in vacuo (water bath: room temperature) and the crude product was obtained as a yellowish oil. The crude product was purified via flash chromatography (loaded as solution in CH₂Cl₂ (6 mL), 30 μm spherical silica gel, Interchim cartridge F0040, 100% CH₂Cl₂ isocratic). (S)-benzyl-4-azido-2-(triflyloxy)-butanoate (2) (2.32 g, 96%) was obtained as an orangeish oil. ¹H-NMR (CDCl₃, 600 MHz) δ=7.41-7.36 (5H, m, Ph-H), 5.30-5.26 (3H, m, Ph-CH₂, BnO₂C—CH), 3.54-3.42 (2H, m, CH₂—CH₂—N₃), 2.28-2.19 (2H, m, CH₂—CH₂—N₃). ¹⁹F-NMR (CDCl₃, 564 MHz) δ=−74.7 (OTf).

(R,R,R)-Bn₃-PCTAZA 8. To a solution of pyclen 7 (273 mg, 1.32 mmol, 1.00 eq.) in anhydrous MeCN (10 mL) was added Cs₂CO₃ (1.51 g, 4.63 mmol, 3.50 eq.) and the suspension was cooled via an ice bath. Following a solution of(S)-benzyl-4-azido-2-(triflyloxy)-butanoate (6) (1.60 g, 4.37 mmol, 3.30 eq.) in anhydrous MeCN (25 mL) was added dropwise over 30 min. The reaction mixture was warmed to room temperature and stirred at this temperature for 2 h. Afterwards the solvent was removed in vacuo and the residue was dissolved in sat. aq. NaHCO₃ solution (30 mL) and CH₂Cl₂ (40 mL). The phases were separated and the aq. phase was extracted with CH₂Cl₂ (3×30 mL). The combined org. phases were dried over Na₂SO₄, the solvent was removed in vacuo and the crude product was obtained as an orange oil. The crude product was purified via flash chromatography (loaded as solution in CH₂Cl₂ (4 mL), 15 μm spherical silica gel, Interchim cartridge F0040, 100% CH₂Cl₂ for 1 cv then gradient to 100% MeCN in 10 cv, 100% MeCN for 3 cv). (R,R,R)-Bn₃-PCTAZA 8 (1.11 g, 98%) was obtained as an orangeish viscous oil. R_{f} (CH₂Cl₂/MeOH/Et₃N 95:4.5:0.5)=0.60. HRMS (ESI) m/z [M+H]⁺ calcd, for C₄₄H₅₂N₁₃O₆⁺: 858.4158, found: 858.4117. ¹H-NMR (CDCl₃, 600 MHz) δ=7.60 (1H, t, ³J_{H,H}=7.67 Hz, C_{pyridine}—Hₚₐᵣₐ to N_{pyridine}), 7.41-7.27 (15H, m, CH₂-Ph-H), 7.10 (2H, d, ³J_{H,H}=7.64 Hz, C_{pyridine}—H meta to N_{pyridine}), 5.22 (2H, d, ²J_{H,H}=12.52 Hz, Ph-CH₂—), 5.18 (2H, d, ²J_{H,H}=12.27 Hz, Ph-CH₂—), 5.03 (2H, s, Ph-CH₂—), 3.94 (2H, d, ²J_{H,H}=11.92 Hz, Py-CH₂—N), 3.75 (2H, d, ²J_{H,H}=11.92 Hz, Py-CH₂—N), 3.52 (2H, m, BnO₂C—CH-next to pyridine), 3.46-3.35 (4H, m, CH₂—CH₂—N₃ sidearms next to pyridine), 3.21-3.14 (3H, m, BnO₂C—CH opposite pyridine, CH₂—CH₂—N₃ sidearm opposite pyridine), 2.90-2.81 (3H, m, CH₂—CH₂—N₃), 2.20-2.13 (3H, m, CH₂—CH₂—N₃), 2.12-1.98 (3H, m, N—CH₂—CH₂—N, N—CH₂—C(H)H—N), 1.93-1.85 (2H, m, N—CH₂—CH₂—N), 1.84-1.78 (1H, m, N—CH₂—C(H)H—N), 1.57-1.52 (1H, m, N—CH₂—C(H)H—N),

1.32-1.26 (1H, m, N—CH₂—C(H)H—N). ¹³C-NMR (CDCl₃, 150 MHz) δ=172.9 (C_{carbonyl} sidearm opposite pyridine), 172.4 (C_{carbonyl} sidearms next to pyridine), 158.3 (quart. C_{pyridine}), 137.6 (quart. C_{phenyl}), 135.9 (Cₚₐᵣₐ to N_{pyridine}), 128.9 (C_{phenyl}), 128.8 (C_{phenyl}), 128.7 (C_{phenyl}), 128.6 (C_{phenyl}), 128.5 (C_{phenyl}), 128.4 (C_{phenyl}), 122.9 (Cₘₑₜₐ to N_{pyridine}), 66.6 (Ph-CH₂ sidearms next to pyridine), 66.3 (Ph- CH₂ sidearm opposite pyridine), 63.8 (BnO₂C—CH—, sidearms next to pyridine), 63.0 (BnO₂C—CH—, sidearm opposite pyridine), 57.9 (pyridine-CH₂—), 52.9 (CH₂—CH₂—N₃), 50.5 (CH₂—CH₂—N₃), 48.4 (CH₂—CH₂—N₃ sidearms next to pyridine), 48.3 (CH₂—CH₂—N₃ sidearm opposite pyridine), 30.0 (N—CH₂—CH₂—N), 29.8 (N—CH₂—CH₂—N). t_{R} (C18 Gravity-SB, method 1): 18.2 min.

Alkyne N-oxide 15. Under nitrogen atmosphere a solution of 3-dimethylamino-1-propyne (13) (2.00 g, 24.0 mmol, 1.00 eq.) in anhydrous CH₂Cl₂ (24 mL) was cooled to 0° C. via an ice bath and subsequently a solution of mCPBA (75% with H₂O, 5.54 g, 24.0 mmol, 1.00 eq) in anhydrous CH₂Cl₂ (24 mL) was added in a single portion. The reaction mixture was warmed to room temperature and stirred at this temperature for 3 h. Afterwards the reaction mixture was passed through a plug of alkaline alumina. The plug was first eluted with CH₂Cl₂ followed by CH₂Cl₂/MeOH 95:5. All volatiles were removed in vacuo (bath temperature at room temperature), the product was dried under high vacuum and 3-dimethylamino-1-propyne N-oxide 15 (1.98 g, 83%) was obtained as a colourless solid. As the product was temperature-sensitive and unstable in its solid form, it was immediately dissolved in MilliQ H₂O to yield a stable 3 M aq. stock solution which was stored at 4° C. HRMS (ESI) m/z [M+H]⁺ calcd, for C₅H₁₀NO⁺: 100.0757, found: 100.0761. ¹H-NMR (D₂O, 300 MHz) δ=4.20 (s, 2H, CH₂), 3.35 (s, 1H, C≡CH), 3.30 (s, 6H, CH₃).

Bn₃-NOx₃-PCTA 10. Under nitrogen atmosphere a mixture of DMF/H₂O (7:1, 1.80 mL) was degassed by purging the solution with nitrogen gas for 15 min under vigorous stirring. Afterwards CuI (8.90 mg, 47.0 μmol, 0.10 eq.) and sodium ascorbate (18.5 mg, 93.0 μmol, 0.20 eq.) were added and the degassing process was allowed to proceed for further 5 min. Following (R,R,R)-Bn₃-PCTAZA 8 (0.40 g, 0.47 mmol, 1.00 eq.) as a solution in DMF (0.25 mL) and an aq. solution of 3-dimethylamino-1-propyne N-oxide (15) (3 M in H₂O, 2.8 mL, 8.39 mmol, 18.0 eq.) were both added in one portion. The reaction mixture was warmed to 55° C. and stirred at this temperature for 65 min. All volatiles were removed in vacuo and the crude product was obtained as a yellowish wax. The crude product was purified via reversed phase flash chromatography (loaded as solution in H₂O (2 mL), 15 μm C18AQ silica, Interchim cartridge F0012, 100% H₂O 5 cv, then gradient to 100% MeCN in 10 cv, 100% MeCN 5 cv). After lyophilization Bn₃-NOx₃-PCTA 10 (392 mg, 73%) was obtained as a beige lyophilisate. HRMS (ESI) m/z [M+H]⁺ calcd, for C₅₉H₇₈N₁₆O₉⁺: 1154.6133, found: 1154.6100. ¹H-NMR (D₂O, 600 MHz) δ=8.31 (1H, s, H_{triazole} sidearm opposite pyridine), 8.11 (2H, s, H_{triazole} sidearms next to pyridine), 7.95 (1H, t, ³J_{H,H}=7.81 Hz, C_{pyridine}—H para to N_{pyridine}), 7.43-7.27 (13H, m, CH₂-Ph-H sidearms next to pyridine, C_{pyridine}—H meta to N_{pyridine}, CH₂-Ph-H sidearm opposite pyridine), 7.20 (2H, dd, ³J_{H,H}=7.71 Hz, CH₂-Ph-H), 7.02 (2H, dd, ³J_{H,H}=7.51 Hz, CH₂-Ph-H), 5.12 (2H, m, Ph-CH₂—, sidearm opposite pyridine), 5.05-4.87 (4H, m, Ph-CH₂—, sidearms next to pyridine), 4.75-4.70 (2H, m, CH₂—CH₂-triazole sidearm opposite pyridine), 4.65-4.60 (4H, m, CH₂-NOx, sidearms next to pyridine), 4.56 (2H, s, CH₂-NOx sidearm opposite pyridine), 4.45-4.22 (m, 4H, CH₂—CH₂-triazole sidearms next to pyridine), 4.00-3.97 (5H, m, BnO₂C—CH—), 3.84 (2H, m, Pyridine-CH₂), 3.45 (2H, m, Pyridine-CH₂), 3.25 (12H, d, ⁴J_{H,H}=7.35 Hz, N(CH₃)₂—O sidearms next to pyridine), 3.22 (6H, d, ⁴J_{H,H}=3.86 Hz, N(CH₃)₂—O sidearm opposite pyridine), 2.94-2.59 (8H, m, N—C₂H₄—N), 2.42-2.36 (4H, m, CH—CH₂), 2.26-2.14 (2H, m, CH—CH₂). ¹³C-NMR (D₂O, 150 MHz) δ=173.0 (C_{carbonyl}), 155.4 (quart. C_{pyridine}), 143.3 (C_{pyridine}—H para to N_{pyridine}), 136.8 (quart. C_{triazole} sidearms opposite pyridine), 136.5 (quart. C_{triazole} sidearms next to pyridine), 135.1 (quart. C_{phenyl} sidearms next to pyridine), 133.7 (quart. C_{phenyl} sidearm opposite pyridine), 129.2, 129.0, 128.9, 128.8, 128.7, 128.6, 128.5, 127.8, 127.5 (C_{phenyl}, C_{triazole}—H), 123.03 (C_{pyridine} meta to N_{pyridine}), 68.6 (Ph-CH₂ next to pyridine), 67.5 (BnO₂C—CH—), 63.8 (CH₂-NOx next to pyridine), 63.7 (CH₂-NOx opposite pyridine), 58.2 (Ph-CH₂ opposite pyridine), 56.5 (N⁺—(CH₃)₂—O sidearms next to pyridine), 56.4 (N⁺—(CH₃)₂—O sidearm opposite pyridine), 52.8 (pyridine-CH₂), 52.0 (pyridine-CH₂), 47.8 (CH₂—CH₂-triazole sidearms next to pyridine), 47.5 (CH₂—CH₂-triazole sidearms opposite pyridine), 42.28 (CH₂—CH₂-triazole), 29.4 (N—C₂H₄—N), 24.1 (N-C₂H₄-N). t_{R} (C18 Gravity-SB, method 1): 11.7 min.

Bn₃-SB₃-PCTA 9. Under nitrogen atmosphere DMF/H₂O 7:1 (8 mL) was degassed by purging the solution with nitrogen gas for 15 min under vigorous stirring. Afterwards CuI (8.90 mg, 47.0 μmol, 0.10 eq.) and sodium ascorbate (18.5 mg, 93.0 μmol, 0.20 eq.) were added and the degassing process was allowed to proceed for further 5 min. Following (R,R,R)-Bn₃-PCTAZA 8 (400 mg, 466 μmol, 1.00 eq.) as a solution in DMF (1.5 mL) and sulfobetaine alkyne 14 (316 mg, 1.64 mmol, 3.30 eq.) were both added in one portion. The reaction mixture was warmed to 50° C. and stirred at this temperature for 41 h during which the reaction solution became dark brown. All volatiles were removed in vacuo and the crude product was obtained as a brownish waxy solid. The crude product was purified via reversed phase flash chromatography (loaded as solution in H₂O (2 mL), 15 μm C18AQ silica, Interchim cartridge F0012, 100% H₂O 5 cv, gradient to 100% MeCN in 10 CV, 100% MeCN 5 cv). After lyophilization Bn₃-SB₃-PCTA 9 (365 mg, 53%) was obtained as colourless lyophilisate. HRMS (ESI) m/z [M+2H]²⁺ calcd, for C₆₈H₉₈N₁₆O₁₅S₃²⁺: 737.3275, found: 737.3293. ¹H-NMR (D₂O, 600 MHz) δ=8.45, 8.43 (s, 2H, H_{triazole} sidearms next to pyridine), 8.26 (s, 1H, H_{triazole} sidearm opposite pyridine), 7.97-7.94 (m, 1H, C_{pyridine}—H para to N_{pyridine}), 7.42-7.02 (m, 17H, CH₂-Ph-H, C_{pyridine}—H meta to N_{pyridine}), 5.11-4.86 (m, 6H, Ph-CH₂), 4.70-4.31 (m, 13H, N⁺(CH₃)₂—CH₂-triazole, Pyridine-CH₂, BnO₂C—CH), 4.00-3.40 (m, 8H, N—CH₂—CH₂—N), 3.37-3.27 (m, 6H, N⁺(CH₃)₂—CH₂—CH₂), 3.09-3.02 (m, 18H, N—(CH₃)₂—O), 2.98-2.60 (m, 12H, CH₂—CH₂—SO₃, CH—CH₂), 2.49-2.20 (m, 12H, CH₂—CH₂—SO₃, CH—CH₂). ¹³C-NMR (CDCl₃, 150 MHz) δ=172.8 (C_{carbonyl}), 170.9 (C_{triazole}—H), 155.6 (HMBC, quart. C_{pyridine}), 143.1 (HSQC, C_{pyridine}—Hₚₐᵣₐ to N_{pyridine}), 135.4, 135.2, 135.1 (quart. C_{triazole} and quart. C_{phenyl}), 129.3, 129.0, 128.9, 128.8, 128.7, 128.6, 128.5, 128.4, 128.2, 127.5 (C_{phenyl}—H and C_{pyridine}—H), 123.1 (C_{phenyl}—H), 120.8 (HSQC, C_{phenyl}—H), 68.4 (Ph-CH₂), 67.5 (BnO₂C—CH—), 67.3 (triazole-CH₂—N⁺(CH₃)₂), 61.8 (pyridine-CH₂), 57.7, 57.6 (N⁺(CH₃)₂—CH₂—CH₂), 50.4, 50.3, 50.2 (N—(CH₃)₂—O), 47.8, 47.7, 47.2 (CH₂—CH₂—SO₃, N—CH₂—CH₂-triazole), 32.7, 29.7, 29.4 (HSQC, N—C₂H₄—N, N—CH₂—CH₂-triazole), 18.3, 18.2 (CH₂—CH₂—SO₃). t_{R} (C18 Gravity-SB, method 1): 13.2 min.

Gd-NOx₃-PCTA 12a. At room temperature Bn₃-NOx₃-PCTA 10 (250 mg, 216 μmol, 1.00 eq.) was dissolved in H₂O (5 mL) and following a solution of NaOH (104 mg, 2.60 mmol, 12.0 eq.) in H₂O (1 mL) was added dropwise. The yellowish solution was stirred at room temperature for 23 h before Dowex® 50W X8 hydrogen form, 100-200 mesh (500 mg) was added and the suspension was stirred until the pH was found to be neutral. The resin was removed via filtration, washed with H₂O (5 mL) and all volatiles were removed in vacuo. The residue was taken up in a solution of GdCl₃ hexahydrate (84.4 mg, 227 μmol, 1.05 eq.) in 1 M aq. NH₄OAc buffer (11 mL, pH 5.5) and the reaction mixture was stirred at room temperature for 3 h. Afterwards all volatiles were removed in vacuo (bath temperature: 55° C.) and the crude product was obtained as a yellowish oily residue. The crude product was purified via reversed phase flash chromatography (loaded as solution in H₂O (1.5 mL), 15 μm C18AQ silica, Interchim cartridge F0012, 3 cv 100% H₂O, then gradient to 100% MeCN in 10 cv, then 100% MeCN holding for 3 cv). After lyophilization Gd-NOx ₃-PCTA 12a (172 mg, 77%) was obtained as a colourless lyophilisate. HRMS (ESI) m/z [M+2H]²⁺ calcd, for C₃₈H₅₈GdN₁₆O₉²⁺: 520.1902, found: 520.1951. t_{R} (C18 Gravity-SB, method 1): 2.3 min.

Gd—SB₃-PCTA 11a. Bn₃-SB₃-PCTA 9 (250 mg, 170 μmol, 1.00 eq.) was dissolved in THF (1 mL) and a solution of NaOH (81.4 mg, 2.04 mmol, 12.0 eq.) in H₂O (5 mL) was added dropwise. The yellowish solution was stirred at room temperature for 23 h before Dowex 50W X8 hydrogen form, mesh 100-200 (350 mg) was added and the suspension was stirred until the pH was found to be neutral. The resin was removed via filtration, washed with THF/H₂O 1:5 (5 mL) and all volatiles were removed in vacuo. The residue was taken up in a solution of GdCl₃ hexahydrate (66.2 mg, 178 μmol, 1.05 eq.) in 1 M aq. NH₄OAc buffer (11 mL, pH 5.5) and the reaction mixture was stirred at room temperature for 1.5 h. All volatiles were removed in vacuo (bath temperature: 55° C.) and the crude product was obtained as a yellowish solid. The crude product was purified via reversed phase flash chromatography (loaded as solution in H₂O (1 mL), 15 μm C18AQ silica, Interchim cartridge F0012, 3 cv 100% H₂O, then gradient to 100% MeCN in 10 cv, then 100% MeCN holding for 3 cv). After lyophilization Gd-SB₃-PCTA 11a (195 mg, 85%) was obtained as a colourless lyophilisate. HRMS (ESI) m/z [M+H+Na]²⁺ calcd, for C₄₇H₇₆GdN₁₆NaO₁₅S₃²⁺: 690.6983, found: 690.6971. t_{R} (C18 Gravity-SB, method 1): 9.2 min.

Eu-NOx₃-PCTA 12b. Bn₃-SB₃-PCTA 9 (14 mg, 12.0 μmol, 1.00 eq.) was dissolved in H₂O (1 mL) and a solution of NaOH (5.8 mg, 145 μmol, 12.0 eq.) in H₂O (0.5 mL) was added dropwise. The yellowish solution was stirred at room temperature for 17 h before Dowex 50W X8 hydrogen form, mesh 100-200 (80 mg) was added and the suspension was stirred until the pH was found to be neutral. The resin was removed via filtration, washed with H₂O (3 mL) and all volatiles were removed in vacuo. The residue was taken up in a solution of EuCl₃ (3.3 mg, 13.0 μmol, 1.05 eq.) in 1 M aq. NH₄OAc buffer (1 mL, pH 5.5) and the reaction mixture was stirred at room temperature for 3 h. All volatiles were removed in vacuo (bath temperature: 55° C.) and the crude product was obtained as a yellowish solid. The crude product was purified via reversed phase flash chromatography (loaded as solution in H₂O (0.2 mL), 15 μm C18AQ silica, Interchim cartridge F0004, 4 cv 100% H₂O, then gradient to 100% MeCN in 10 cv, then 100% MeCN holding for 3 cv). After lyophilization Eu-NOx₃-PCTA 12b (6 mg, 48%) was obtained as a colourless lyophilisate. HRMS (ESI) m/z [M+2H]²⁺ calcd, for C₃₈H₅₉EuN₁₆O₉²⁺: 518.1926, found: 518.1952. t_{R} (C18 Gravity-SB, method 1): 2.0 min.

Eu—SB₃-PCTA 11b. Bn₃-SB₃-PCTA 9 (19.5 mg, 13.0 μmol, 1.00 eq.) was dissolved in H₂O (1 mL) and a solution of NaOH (6.4 mg, 159 μmol, 12.0 eq.) in H₂O (0.5 mL) was added dropwise. The yellowish solution was stirred at room temperature for 18 h before Dowex® 50W X8 hydrogen form, mesh 100-200 (80 mg) was added and the suspension was stirred until the pH was found to be neutral. The resin was removed via filtration, washed with H₂O (3 mL) and all volatiles were removed in vacuo. The residue was taken up in a solution of EuCl₃ (3.6 mg, 14.0 μmol, 1.05 eq.) in 1 M aq. NH₄OAc buffer (1 mL, pH 5.5) and the reaction mixture was stirred at room temperature for 3.5 h. All volatiles were removed in vacuo (bath temperature: 55° C.) and the crude product was obtained as a yellowish solid. The crude product was purified via reversed phase flash chromatography (loaded as solution in H₂O (0.2 mL), 15 μm C18AQ silica, Interchim cartridge F0004, 4 cv 100% H₂O, then gradient to 100% MeCN in 10 cv, then 100% MeCN holding for 3 cv). After lyophilization Eu-SB₃-PCTA 11b (10 mg, 56%) was obtained as a colourless lyophilisate. HRMS (ESI) m/z [M+2H]²⁺ calcd, for C₄₇H₇₇EuN₁₆O₁₅S₃²⁺: 677.2059, found: 677.2052. t_{R} (C18 Gravity-SB, method 1): 9.3 min.

### Kinetic and Thermodynamic Stability of Zwitterionic Gd(III) Complexes

Kinetic inertness is a key determinant of the in vivo stability of GBCAs, as it governs the resistance of the complex to decomplexation processes under physiological conditions. Dissociation of macrocyclic lanthanide complexes occurs via protonation of the complex, leading to the formation of a diprotonated intermediate, which then undergoes loss of Gd(III). This process of decomplexation can be monitored by spectrophotometric quantification of free Gd(III) using complexometric titration with Arsenazo III. The stability of Gd—SB₃-PCTA 11a and Gd-NOx₃ -PCTA 12a was assessed in aqueous solution at pH 1.2 and directly compared to that of gadopiclenol 3 as well as Gd-DOTA 2 (gadoteric acid) under identical conditions.

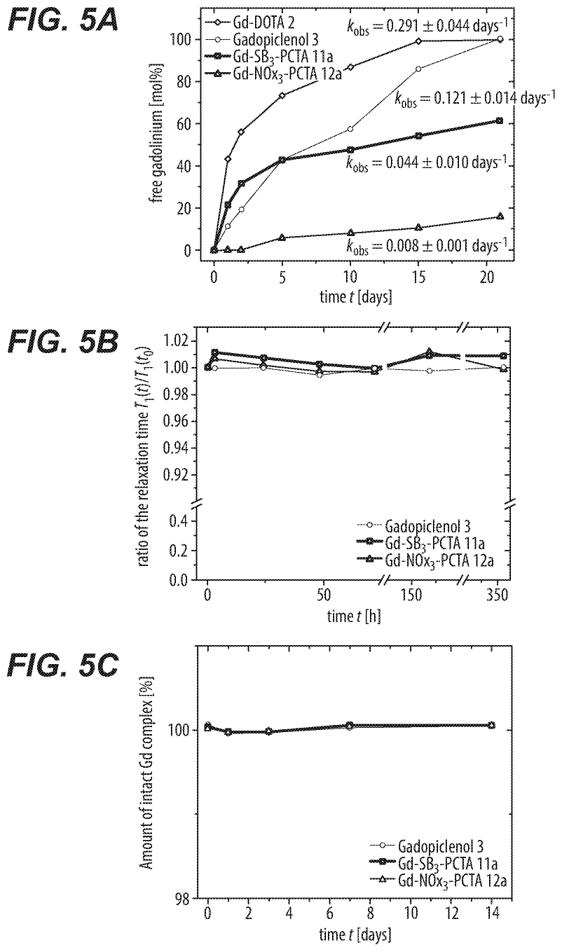

As previously reported by Robic et al., gadopiclenol 3 is significantly more stable than the current gold standard Gd-DOTA 2 and far more stable than the more labile GBCAs gadodiamide and gadobutrol. (C. Robic, M. Port, O. Rousseaux, S. Louguet, N. Fretellier, S. Catoen, C. Factor, S. Le Greneur, C. Medina, P. Bourrinet, I. Raynal, J. M. Idee and C. Corot, Invest. Radiol., 2019, 54, 475-484.) This stability trend was confirmed for gadopiclenol 3 and Gd-DOTA 2 in experiments as depicted in FIG. 5. Notably, the zwitterionic complexes Gd—SB₃-PCTA 11a and Gd-NOx₃-PCTA 12a are more stable under acidic conditions than gadopiclenol 3. After 21 days, gadopiclenol 3 was completely dissociated, while Gd—SB₃-PCTA 11a released only 62% and Gd-NOx₃-PCTA 12a just 16% of its Gd(III) content (FIG. 4).

The dissociation data of the Gd(III) complexes were evaluated under the assumption that both zwitterionic and non-zwitterionic complexes follow pseudo-first order dissociation kinetics. Linearisation according to the equation In (Aₜ/A₀)=−k_{obs}·t followed by linear regression, gave the dissociation rate constants k_{obs}. The parameter Aₜ denotes the concentration of dissociated complex at time t and A₀ the initial concentration of the complex.

Comparison of the observed dissociation rate constants and the total amount of Gd(III) released after 21 days revealed significant differences in the kinetic stability of the complexes. The most stable compound, Gd-NOx₃-PCTA 12a had a dissociation rate that was more than 36-fold lower than that of the gold standard GBCA Gd-DOTA 2 and 15-fold lower than that of gadopiclenol 3. Notably, Gd-NOx₃-PCTA 12a also dissociated 5.5 times more slowly than the sulfobetaine-analogue Gd—SB₃-PCTA 11a, highlighting a substantial difference in complex stability among zwitterionic complexes. However, both new complexes Gd—SB₃-PCTA 11a and Gd-NOx₃-PCTA 12a are exceptionally stable under acidic conditions with dissociation rates 3 or 15 times lower respectively than that of gadopiclenol 3.

These findings reveal a stabilizing effect of zwitterionic modifications on the kinetic inertness of the complexes.

In contrast to the in vitro evaluation of kinetic stability, the assessment of thermodynamic stability of GBCAs is more challenging, as it cannot be easily extrapolated to in vivo conditions due to the complex composition of biological media, containing numerous competing endogenous complex ligands and metal cations (e.g. Fe(III), Ca(II), Zn(II), Cu(II). A robust and widely accepted method for evaluating GBCA stability under physiologically relevant conditions involves transmetalation with Zn(II) in phosphate buffer. The zwitterionic complexes Gd-SB₃-PCTA 11a and Gd-NOx₃-PCTA 12a as well as gadopiclenol as non-zwitterionic reference were challenged with excess Zn(II) in phosphate-buffered saline (PBS, pH 7.4) at 37° C. Longitudinal relaxation rates were monitored at the following time points: 0 h, 3 h, 1 day, 3 days, 7 days and 15 days. It is important to note that the Zn(II) solution must be freshly prepared with ZnCl₂ and added immediately to the chelator solutions to avoid the formation of zinc phosphate precipitates.³⁰ The zwitterionic complexes Gd—SB₃-PCTA 11a and Gd-NOx₃-PCTA 12a, as well as gadopiclenol 3, showed no signs of instability as the measured longitudinal relaxation rates remained constant throughout the observation period of 15 days. These results are consistent with previous studies of gadopiclenol¹⁴ and suggest that Gd—SB₃-PCTA 11a and Gd-NOx₃-PCTA 12a are at least as stable as gadopiclenol 3 against transchelation.

These observations were further confirmed by a stability assay in human serum at 37° C. All Gd(III) complexes were incubated for 15 days and gadolinium release was quantified at 0 h, 1 day, 3 days, 7 days and 15 days by complexometric titration with Arsenazo III. No loss of Gd(III) was observed for any of the zwitterionic complexes Gd—SB₃-PCTA 11a and Gd-NOx₃-PCTA 12a or for gadopiclenol 3.

### Relaxivity at 1.4 T and 7 T

The relaxivity of GBCAs depends on several factors, including hydration number q, the mean residence time of inner-sphere water molecules, the electronic relaxation time and the rotational correlation time of the complex. Relaxivity arises from the combined contributions of inner-sphere (IS) and outer-sphere (OS) interactions, as described by the following equation:

$${R}_{1} = {R}_{1 , inner - sphere} + {R}_{1 , outer - sphere} .$$

Here R_{1,inner-sphere} refers to the longitudinal relaxation rate originating from inner-sphere interactions and R_{1,outer-sphere} accounts for the contribution from outer-sphere interactions. The inner-sphere component refers to the relaxation of water molecules directly bound the Gd(III) ion, whereas the outer-sphere contribution results from transient interactions between the complex and bulk water molecules not directly bound to the metal centre. An effective strategy to enhance the longitudinal relaxation rate R₁ is to increase the inner-sphere contribution by raising the hydration number q.

According to the Solomon-Bloembergen-Morgan (SBM) theory, R₁ is directly proportional to the hydration number q:

$${R}_{1 , inner sphere} = \frac{{q}_{Gd} [ GBCA ]}{[ {H}_{2} O ]} \frac{1}{{T}_{1 m} + {τ}_{m}}$$

where q_{Gd} refers to the number of inner-sphere water molecules coordinated per Gd(III), [GBCA] is the concentration of the contrast agent, [H₂O] is the bulk water concentration, T₁ₘ is the longitudinal relaxation time of the inner-sphere water protons and τₘ is the mean residence time of these coordinated water molecules. (L. M. De Leon-Rodriguez, A. F. Martins, M. C. Pinho, N. M. Rofsky and A. D. Sherry, J. Magn. Reson. Imaging, 2015, 42, 545-565.)

The common macrocyclic GBCA Gd-DOTA 2 (DOTAREM) coordinates only one inner-sphere water (q=1). Many chelators with an increased hydration number of q=2 have a higher relaxivity, but reduced complex stability compared to Gd-DOTA 2. An exception is gadopiclenol 3, a macrocyclic GBCA based on a chiral PCTA-scaffold with q=2. It combines high relaxivity and high stability.

Another key factor contributing to the overall relaxivity of a GBCA is the outer-sphere component, which can be modulated by parameters such as molecular size, rotational correlation time and electronic relaxation time. As small molecules, GBCAs typically exhibit rapid rotational diffusion, which limits their relaxivity. The outer-sphere contribution can be enhanced by increasing the hydrodynamic size of the complex, thereby reducing its rotational correlation time and resulting in higher relaxivity.

Relaxometric studies of the two zwitterionic GBCAs Gd—SB₃-PCTA 11a and Gd-NOx₃-PCTA 12a were performed at two different and clinically relevant field strengths (1.4 T and 7 T). The longitudinal relaxivity r₁ of both zwitterionic complexes, in comparison with gadopiclenol as a reference, was investigated at a field strength of 1.4 T (60 MHz, 37° C., water) using a standard inversion-recovery pulse sequence. The spin lattice relaxation time (T₁) was determined and plotted as the reciprocal (1/T₁) against the concentration of the respective complexes. The concentrations of the complexes were precisely determined by inductively coupled plasma mass spectrometry (ICP-MS).

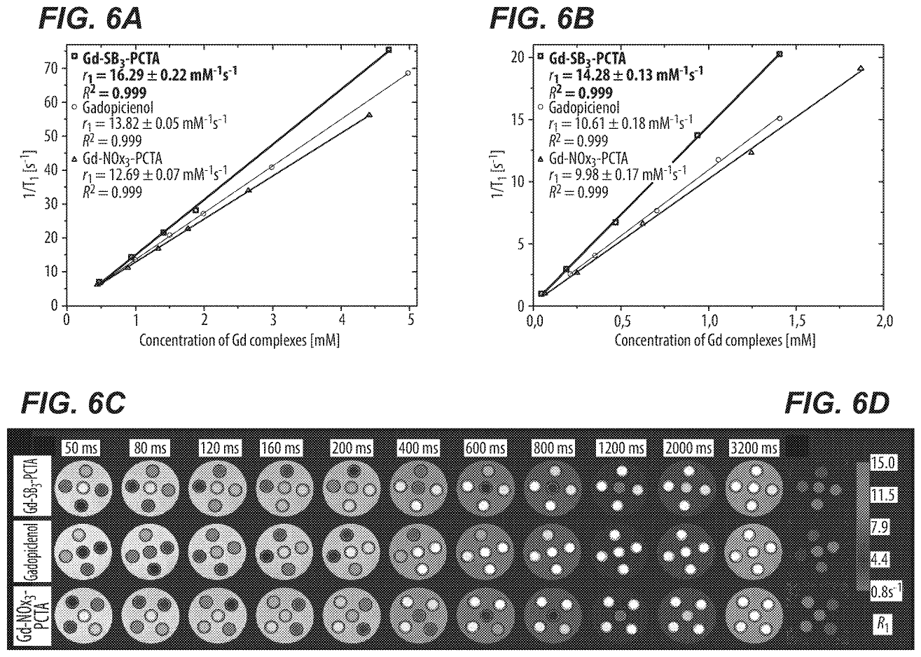

For Gd—SB₃-PCTA 11a a longitudinal relaxivity of r_{1, 1.4 T}=16.29±0.22 mM ⁻¹ s⁻¹ was observed, representing an 18% increase compared to gadopiclenol (r_{1, 1.4T}=13.82±0.05 mM⁻¹ s⁻¹). Gd-NOx₃-PCTA 12a had a slightly lower relaxivity of r_{1, 1.4T}=12.69±0.07 mM⁻¹ s⁻¹, representing an 8% decrease compared to gadopiclenol (FIG. 6).

To evaluate the field dependence of relaxivity, additional measurements were conducted at 7 T (300 MHz, room temperature, water), a magnetic field strength increasingly relevant in preclinical and high-resolution clinical imaging. These measurements were performed using a preclinical MR imager by measuring a slice-selective 2D inversion recovery spin echo sequence with inversion times ranging from 50 ms to 3200 ms (FIG. 6).

Both zwitterionic complexes showed a magnetic field dependence comparable to gadopiclenol, maintaining high relaxivity values even at 7 T. For Gd—SB₃-PCTA 11a a longitudinal relaxivity of r_{1, 7T}=14.28±0.22 mM⁻¹ s⁻¹ was measured, while Gd-NOx₃-PCTA 12a exhibited a relaxivity of r_{1, 7T}=9.98±0.17 mM⁻¹ s⁻¹. Compared to the values at 1.4 T this corresponds to a field dependent relaxivity reduction of 12% for Gd—SB₃-PCTA 11a, 23% for gadopiclenol 3 and 21% for Gd-NOx₃-PCTA 12a (Table 1).

TABLE 1
Molecular weights, longitudinal relaxivity (r₁) values at 1.4 T and 7 T and q values
for Gd-SB₃-PCTA 11a, Gd-NOx₃-PCTA 12a, gadopiclenol 3, Gd-PCTA and Gd-DOTA 2.

| Parameter | Gd-SB₃-PCTA | Gd-NOx₃-PCTA | Gadopiclenol | Gd-PCTA | Gd-DOTA |
|---|---|---|---|---|---|
| M [g · mol⁻¹] | 1357.39 | 1039.23 | 970.10 | 534.63 | 558.64 |
| r_{1, 1.4T} [mM⁻¹ s⁻¹] | 16.29 ± 0.22^{(a)} | 12.69 ± 0.07^{(a)} | 13.82 ± 0.05^{(a)} | 4.9^{(d, e)} | 3.3^{(d, e)} |
| r_{1, 7T} [mM⁻¹ s⁻¹] | 14.28 ± 0.13^{(b)} | 9.98 ± 0.17^{(b)} | 10.61 ± 0.18^{(b)} (10.68^{(f)}) | — | — |
| q | 2.08^{(c)} | 2.23^{(c)} | 2^{(e)} | 2^{(e)} | 1^{(e)} |

^{(a)}H₂O, 37° C.
^{(b)}H₂O, room temperature
^{(c)}determined via luminescence lifetime measurements of the Eu(III) complexes
^{(d)}1.5 T, 37° C.
^{(e)}Napolitano et al.
^{(f)} Robic et al.
R. Napolitano, N. Guidolin, M. Boccalon, A. Fringuello Mingo, S. Colombo Serra, F. Buonsanti, R. Fretta, N. Demitri, A. Benyei, M. Botta, G. B. Giovenzana, F. Tedoldi and Z. Baranyai, Adv. Sci., 2025, 12, e2415321.
C. Robic, M. Port, O. Rousseaux, S. Louguet, N. Fretellier, S. Catoen, C. Factor, S. Le Greneur, C. Medina, P. Bourrinet, I. Raynal, J. M. Idee and C. Corot, Invest. Radiol., 2019, 54, 475-484.

In summary, Gd—SB₃-PCTA 11a has a consistently higher relaxivity than the benchmark GBCA gadopiclenol 3 at a magnetic field strength of 1.4 and 7 T. Its superior performance is attributed to enhanced hydration caused by the sulfobetaine side chains, which appear to provide more effective kosmotropicity than the polar isoserinol sidechains present in gadopiclenol 3. This gain in hydration likely contributes not only to a larger hydrodynamic radius, thereby slowing rotational diffusion, but may also promote dynamic equilibria involved in inner-sphere und outer-sphere processes.

### Relaxivity Measurements at 1.4 T

The determination of the longitudinal relaxivity at 60 MHz (1.4 T) was conducted based on the spin lattice relaxation time (T₁) using a Bruker Minispec mq60 analyzer at 37° C. T₁ measurements were performed using the standard inversion recovery pulse sequence) (180°-τ−90° at a temperature of 37° C.±0.1° C. For each measurement 20 unevenly spaced data points were acquired after inversion times between 5 ms and 400 ms. r₁ was obtained by plotting the inverse relaxation time 1/T₁ versus the concentration of the complexes and slope determination via the linear regression function in OriginLab 2022. The concentration of the complexes was determined via ICP-MS of the highest sample concentration.

### Relaxivity Measurements at 7 T

Relaxation time measurements at 7 T were conducted using a preclinical MRI scanner (Bruker Biospec 70/30, Ettlingen) at room temperature with a volume receive coil of an inner diameter of 40 mm. Five PCR tubes of 320 μL volume were positioned in a holder within a 50 ml Falcon tube filled with water, aligned parallel to the magnet bore. All imaging sequences utilized transversal orientation.

Following an initial survey scan, two distinct T₁-measurement protocols were implemented. A 2D Look Locker EPI-based sequence provided quick qualitative visual T₁ inspection within 4 minutes. This protocol employed inversion times (TI) ranging from 20 to 770 ms in 50 ms increments, with TR 5000 ms and TE 9.9 ms. K-space acquisition utilized six segments with 8 averages, 10° RF pulses, and a 200 kHz readout bandwidth. Imaging parameters included a 32 mm field of view, 128×128 matrix, and 1.2 mm slice thickness.

Subsequently, a slice-selective 2D inversion recovery spin echo sequence was acquired over approximately 2 hours. This protocol incorporated eleven inversion times (50, 80, 120, 160, 200, 400, 600, 800, 1200, 2000, and 3200 ms) with TE 7.0 ms, TR 5000 ms, and an 80 kHz readout bandwidth. Three slices of 1 mm thickness were obtained with 0.5 mm inter-slice gaps and 30 mm field of view with a 128×128 matrix.

Relaxation time analysis was conducted using qMapIt, an in-house quantification software that extends ImageJ functionality. Inversion recovery datasets underwent model function fitting:

$${Signal}_{TI} = A \cdot \frac{1 + {e}^{( - {R}_{1} \cdot TI )} - 2 \cdot {e}^{( - {R}_{1} \cdot TR )}}{1 - 2 \cdot {e}^{( - {R}_{1} \cdot TR )}} \qquad ( Equation 4 )$$

with T₁=R₁⁻¹ via a nonlinear least-squares Dog-Leg algorithm, generating amplitude A and relaxation time T₁ maps. The inversion recovery data was smoothed before fitting with a Median filter of 0.5 pixel to reduce Gibbs-ringing.

Automated contour detection identified individual tube boundaries, with segmented regions stored as regions of interest (ROIs) for subsequent quantitative analysis. The complete analytical workflow was orchestrated through a Python script executed within a Jupyter notebook environment, which seamlessly integrated Fiji and qMapIt operations via PyImageJ.

### In Vivo Gadolinium Kidney Retention

In a first attempt to assess the impact of zwitterionic modifications on in vivo Gd(III) retention, mice were treated with the most promising zwitterionic GBCA Gd—SB₃-PCTA 11a and gadopiclenol 3 as a current clinical reference, in analogy to an in vivo study design reported by Di Gregorio et al. (E. Di Gregorio, R. Iani, G. Ferrauto, R. Nuzzi, S. Aime and E. Gianolio, J Trace Elem Med Biol, 2018, 48, 239-245) Animals received ten consecutive doses of 0.1 mmol·kg⁻¹ corresponding to a cumulative dose of 1 mmol·kg⁻¹. After a 17-day clearance phase following the last administration organs were harvested and analyzed. The residual renal Gd(III) content was therefore quantified by ICP-MS, revealing amounts of 5.89±0.26 nmol·g⁻¹ Gd(III) for the reference compound gadopiclenol 3 and 2.97±0.62 nmol g⁻¹ for the zwitterionic derivative Gd—SB₃—

### In Vivo Kidney Retention Study

For in vivo evaluation of renal gadolinium retention male C57BL/6J mice (n=2, 10-12 weeks, 25-30 g) received 10 consecutive intravenous injections of gadopiclenol 3 and Gd—SB₃-PCTA 11a via the tail vein at 0.1 mmol·kg⁻¹ each (100 μL, saline, pH 7.4) over 10 days. Seventeen days after the final administration, animals were euthanised, organs were harvested and kept frozen until organ digestion and ICP-MS analysis was performed as described in the following.

### Kidney Digestion and ICP-MS Analysis

Kidneys (n=2) were transferred into pre-cleaned tubes, weighed and spiked with dysprosium as internal standard (THERMO FISHER SPECPURE™, Dy plasma standard, 1008±8 μg/mL) to reach 100 μg/L in the final solution used for ICP-MS. For digestion, 2 mL of nitric acid (ROTIPURAN® Supra, 69%) were added and samples were incubated at 37° C. for 18 h. The digests were diluted to 10 mL with MilliQ H₂O, followed by a 1:2 dilution. To remove residual particulates, the solutions were centrifuged at 3500×g for 15 min, decanted and subsequently analysed by ICP-MS in triplicates under the same conditions as described before.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain using no more than routine experimentation, many equivalents to the specific embodiments of the invention described herein.

Such equivalents are intended to be encompassed by the following claims.

### REFERENCES

| Year | DOI | Title | Authors | Citation |
|---|---|---|---|---|
| 2014 | 10.1002/ | Synthesis of 1,4,7,10-tetra- | Kriemen et | Chem. Asian J. 2014, 9 (8), |
| | asia.201402250 | azacyclododecan-1,4,7,10-tetra- | al. | 2197-2204. |
| | | azidoethylacetic acid (DOTAZA) and | | |
| | | related “clickable” DOTA | | |
| | | derivatives. | | |
| 2017 | | Poster - P342 | Henry et al. | J. Label Compd Radiopharm, |
| | | Targeted Zwitterionic Radioligands | | 2017: 60 (Suppl. 1): S111-S640 |
| | | for Improved Molecular Imaging | | |
| 2019 | 10.1021/ | Nonradioactive Cell Assay for the | Holzapfel et | J. Med. Chem., 2019, 62, 10912- |
| | acs.jmedchem.9b01606 | Evaluation of Modular Prostate- | al. | 10918 |
| | | Specific Membrane Antigen | | |
| | | Targeting Ligands via Inductively | | |
| | | Coupled Plasma Mass Spectrometry | | |
| 2022 | 10.1002/ | Solution Structure and Relaxivity of | Holzapfel et | Eur. J. Inorg. Chem. 2022, |
| | ejic.202200432 | Ln-DOTXAZA Derivatives. | al. | e202200432 |
| 2015 | 10.1002/ | Synthesis and Structural Analysis of | Kriemen | Eur. J. Inorg. Chem. 2015, 5368- |
| | ejic.201500789 | 1,4,7,10-tetra-azacyclododecan- | et al. | 5378 |
| | | 1,4,7,10-tetra-azidoethylacetic | | |
| | | acid (DOTAZA) Complexes | | |
| 2023 | 10.1002/ | Synthesis of Modular | Outzen | ChemMedChem 2023, |
| | cmdc.2003000112 | Desferriozamine Analogues and | et al. | e202300112 |
| | | Evaluation of Derivatives for | | |
| | | Zirconium Complexation | | |
| 2019 | DOI: 10.1097/ | Physicochemical and | Robic et al. | J. Invest Radiol 2019; 54: 475- |
| | RLI.0000000000000563 | Pharmacokinetic Profiles of | | 484 |
| | | Gadopiclenol: A New Macrocyclic | | |
| | | Gadolinium Chelate With High T1 | | |
| | | Relaxivity | | |
| 2019 | DOI: 10.1002/ | Efficient Synthesis of a Family of | Leygue et al. | Eur. J. Org. Chem. 2019; 2899- |
| | ejoc.201900280 | Bifunctional Chelators Based on | | 2913 |
| | | the PCTA[12] Macrocycle | | |
| | | Suitable for Bioconjugation | | |
| 2026 | Submitted | Zwitterionic MRI contrast agents | Spickschen | Journal of Materials Chemistry |
| | | with enhanced relaxivity, | et al. | B |
| | | stability, and reduced renal | | |
| | | retention | | |

The contents of all patent, patent applications, and publications cited herein are incorporated herein by reference in their entireties.

1. A zwitterionic metal chelator complex comprising a metal chelator having one or more zwitterionic groups and a metal or metal isotope selected from the group consisting of a radionuclide, a label, a paramagnetic metal and a heavy metal.

2. The zwitterionic metal chelator complex according to claim 1, wherein the metal chelator is a derivative of PCTA, amide-PCTA, or PCTP.

3. The zwitterionic metal chelator complex according to claim 1, further comprising one or more targeting vectors.

4. The zwitterionic metal chelator complex according to claim 3, wherein the one or more targeting vectors are cRGD, KUE, a GPI-derivative, PSMA-617, FAPI, octreotide, a GPI-derivative, a bombesin analog, or a homo- or hetero-dimer formed from their combination.

5. The zwitterionic metal chelator complex according to claim 1, wherein the metal chelator is a derivative of PCTA and the metal or metal isotope is Gd or Gd³⁺, Pb, Mn, Zr, Cu, Ga, In, Y, Gd, Lu, c or Tb.

6. The zwitterionic metal chelator complex according to claim 1, wherein the metal chelator is a derivative of amide-PCTA and the metal or metal isotope is Gd, Gd³⁺, Pb, Mn, Zr, Cu, Ga, In, Y, Gd, Lu, c or Tb.

7. The zwitterionic metal chelator complex according to claim 2, wherein the metal chelator is a derivative of PCTA and wherein the zwitterionic metal chelator of the zwitterionic metal chelator complex has the formula: 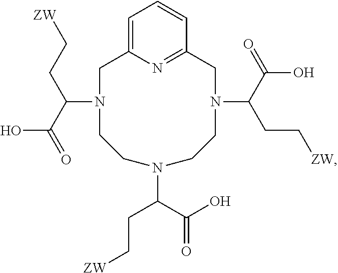
wherein ZW represents a zwitterionic group.

8. The zwitterionic metal chelator complex according to claim 2, the metal chelator is a derivative of amide-PCTA, wherein the zwitterionic metal chelator of the zwitterionic metal chelator complex has the formula: 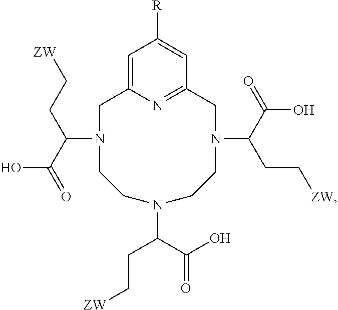
wherein ZW represents a zwitterionic group, wherein R is a targeting vector or a reactive group.

9. The zwitterionic metal chelator complex according to claim 2, wherein the metal chelator is a derivative of amide-PCTA, wherein the zwitterionic metal chelator of the zwitterionic metal chelator complex has the formula: 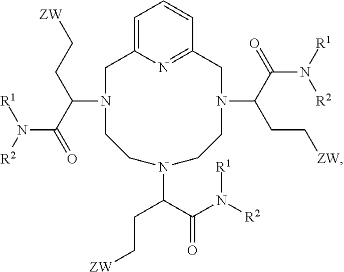
wherein ZW represents a zwitterionic group, and R¹ and R² are independently H, alkyl or aryl.

10. The zwitterionic metal chelator complex according to claim 2, wherein the metal chelator is a derivative of amide-PCTA, wherein the zwitterionic metal chelator of the zwitterionic metal chelator complex has the formula: 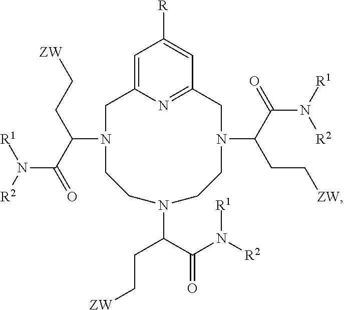
wherein ZW represents a zwitterionic group, R is a targeting vector or a reactive group, and R¹ and R² are H, alkyl or aryl.

11. The zwitterionic metal chelator complex according to claim 2, wherein the metal chelator is a derivative of PCTP and the metal or metal isotope is Pd, Pb, Mn, Zr, Cu, Ga, In, Y, Gd, Lu, Ac or Tb.

12. The zwitterionic metal chelator complex according to claim 2, wherein the zwitterionic metal chelator of the zwitterionic metal chelator complex has the formula:

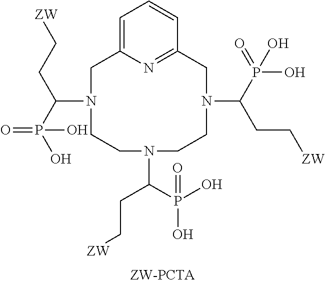

13. An imaging agent comprising a zwitterionic metal chelator complex according to claim 1.

14. A method of imaging cells, tissues, or organs, the method comprising:
(a) contacting cells with an imaging agent according to claim 13; and
(b) imaging the cells, tissues, or organs using positron emission tomography (PET), single-photon emission computerized tomography (SPECT), or magnetic resonance imaging (MRI).

15-18. (canceled)

19. A therapeutic agent comprising a zwitterionic metal chelator complex according to claim 1 and a pharmaceutically acceptable carrier or excipient.

20. A method of treating a cancerous condition in a subject in need thereof, the method comprising:
contacting cancer cells in the subject with an effective amount of a therapeutic agent according to claim 19,
wherein the metal atom complexed to the zwitterionic metal chelator is:
a radioactive metal isotope known to emit ionizing radiation that results in the death of cells that take up the analogs;
or a non-radioactive metal that is capable of releasing cytotoxic radiation upon irradiation with alpha emission, beta emission, neutron capture, or a combination thereof.

21. The method of treating a cancerous condition in a subject in need thereof according to claim 18, wherein the metal atom complexed to the zwitterionic metal chelator is a non-radioactive metal that is capable of releasing cytotoxic radiation upon irradiation with alpha emission, beta emission, neutron capture, or a combination thereof; the method further comprising a step of irradiating the tumor cells using alpha emission, beta emission, neutron capture, or a combination thereof.

22. The method of treating a cancerous condition in a subject in need thereof according to claim 20, wherein the cancer cells are adult solid tumor cells or pediatric solid tumor cells.

23. The method of treating a cancerous condition in a subject in need thereof according to claim 20, wherein the cancer cells are melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

24. (canceled)

25. (canceled)

26. A method of treating a non-cancerous condition in a subject in need thereof, the method comprising:
administering to the subject an effective amount of a therapeutic agent according to claim 20,
wherein the metal atom complexed to the zwitterionic metal chelator is:
a radioactive metal isotope known to emit ionizing radiation that results in a therapeutic effect on the subject;
or a non-radioactive metal that is capable of releasing therapeutic radiation upon irradiation with alpha emission, beta emission, neutron capture, or a combination thereof.

27. The method of treating a non-cancerous condition in a subject in need thereof according to claim 26, wherein the non-cancerous condition is a musculoskeletal disorders or a tissue hypertrophy disorder.

28. The method of treating a non-cancerous condition in a subject in need thereof according to claim 26, wherein the subject is a human.

29. A diagnostic agent comprising a zwitterionic metal chelator complex according to claim 1 and a pharmaceutically acceptable carrier or excipient.

30.-34. (canceled)

35. A radiosurgical method for treating a patient body, the method comprising:
receiving a desired lesion pattern and planned radiation distribution;
administering an effective amount of a diagnostic agent according to claim 29 to the subject to effectively image the desired lesion pattern;
performing surgery on the desired lesion pattern to treat the patient body.

36. The radiosurgical method according to claim 35, wherein the zwitterionic metal chelator of the diagnostic agent further comprises one or more targeting vectors
wherein the one or more targeting vectors are cRGD, PSMA-617, FAPI, octreotide, a bombesin analog, or a homo- or hetero-dimer formed from their combination.

37. The radiosurgical method according to claim 35, wherein the desired lesion pattern is received from a user interface of a treatment planning module.

38. The radiosurgical method according to claim 37, wherein the treatment planning module is pre-programmed with specifications for various disease states and cancerous conditions.

39. The radiosurgical method according to claim 37, wherein the treatment planning module identifies lesion patterns for various disease states and cancerous conditions using artificial intelligence data.

40. The radiosurgical method according to claim 37, wherein the surgery is performed using a sterotactic radiosurgical system.

41. A method of treating a cancer by administering an effective amount of a therapeutic agent according to claim 19, wherein the method comprises a step to diagnose the cancer and a step of administering the therapeutic agent to a subject determined to be in need thereof;
wherein the step to diagnose the cancer comprises:
contacting cells, tissues or organs of a subject with an imaging agent,
imaging the cells, tissues, or organs of the subject using positron emission tomography (PET), single-photon emission computerized tomography (SPECT), or magnetic resonance imaging (MRI), and
diagnosing the cancer in the cells tissues, or organs of the subject based on imaging data collected;
and wherein the metal atom complexed to the zwitterionic metal chelator is:
a radioactive metal isotope known to emit ionizing radiation that results in the death of cells that take up the analogs;
or a non-radioactive metal that is capable of releasing cytotoxic radiation upon irradiation with alpha emission, beta emission, neutron capture, or a combination thereof.


SEQUENCE LISTING (6 sequences)

SEQ ID NO: 1 - protein, 9 aa, Homo sapiens
CGQKRTRGC

SEQ ID NO: 2 - protein, 12 aa, Homo sapiens
HTMYYHHYQHHL

SEQ ID NO: 3 - protein, 9 aa, Homo sapiens
CRKRLDRNC

SEQ ID NO: 4 - protein, 6 aa, Homo sapiens
CSPGAK

SEQ ID NO: 5 - protein, 8 aa, Homo sapiens
FCFWKTCT

SEQ ID NO: 6 - protein, 14 aa, Homo sapiens
XQRLGNQWAVGHLM